(19) United States
(12) Patent Application Publication
(10) Pub. No.: US 2005/0266428 A1
(43) Pub. Date: Dec. 1, 2005

(54) Purification methods

(75) Inventors: Tzong-Hsien Lee, Clayton (AU); Patrick Perlmutter, Ashburton (AU); Marie-Isabel Aguilar, North Warrandyte (AU)

Correspondence Address: LADAS & PARRY, 26 WEST 61ST STREET, NEW YORK, NY, 10023, US

(73) Assignee: MONASH UNIVERSITY

(21) Appl. No.: 11/032,698

(22) Filed: Dec. 29, 2004

Related U.S. Application Data
(63) Continuation of application No. PCT/AU03/00850, filed on Jul. 2, 2003.

(30) Foreign Application Priority Data
Jul. 3, 2002 (AU) PS3349

(51) Int. Cl. C12Q 1/68; C07H 21/00; G01N 33/53

(52) U.S. Cl. 435/6; 435/7.1; 536/25.4; 530/412

(57) ABSTRACT

The present invention relates to methods of processing of chemical samples containing compounds or molecular complexes using a binding material comprising a method of separating a compound or molecular complex on the basis of the ability of the compound or molecular complex to associate with a binding material, from compounds or molecular complexes having different association characteristics, said method comprising: (a) bringing a sample containing said compound or molecular complex into contact with a binding material, the binding material comprising: (i) a support, (ii) at least one terminal moiety selected from the group consisting of lecithins, lysolecithins, cephalins, sphingomyelin, cardiolipin, glycolipids, gangliosides, cerebrosides and phospholipids, and
(iii) at least one linker of the formula (I) wherein R₁ and R₂ may be the same or different and are independently selected from the group consisting of: H, OH, C₁-C₆ alkyl, C₂-C₆ alkenyl, halogen, C₁-C₆ alkoxy, C₂-C₆ alkenyloxy and aryloxy, or R₁ or R₂ when taken together with an R₁ or R₂ on an adjacent linker forms a group of formula —O—, wherein said group —O— connects the silicon atom of the linker to the silicon atom of the adjacent linker, R₃ and R₄ may be the same or different and are independently selected from the group consisting of H, C₁-C₆ alkyl, C₂-C₆ alkenyl and halogen, R₅ is H or C₁-C₆ alkyl, R₆ is H, C₁-C₆ alkyl, C₂-C₆ alkenyl, aryl and heteroaryl, X is O or S, n is an integer from 0 to 10, the terminal moiety being bound to the support via at least one linker, (b) treating the product of step (a) to separate the components of the sample on the basis of their ability to associate with the binding material.

### FIELD OF THE INVENTION

The present invention relates to methods of utilising a binding material in the processing of samples containing chemical compounds or molecular complexes. Such processing typically utilises the ability of the binding material to preferentially bind to the compound or molecular complex of interest and, therefore, relies on the properties of the binding material. The invention therefore includes processes such as detection of compounds or molecular complexes such as in high throughput screening methodologies using array technology. The invention also relates to methods of increasing the purity of compounds or molecular complexes in a sample. In a preferred embodiment, this leads to the purification of a sample containing a compound or molecular complex such as in the purification of samples into their component parts. The methods also include the analysis of a sample in order to determine its components. Many of the existing techniques are not suitable for use with many compounds as they are either too expensive or destroy the structural integrity of the materials in the sample to be analysed such as when proteins or peptides are analysed. The applicants have now found a binding material that is well suited to these applications, especially where the sample contains proteins, especially membrane proteins.

### BACKGROUND OF THE INVENTION

With the recent completion of the human and other genome-sequences, there has been a frenzy of activity directed towards the development of new technologies aimed at exploiting the knowledge provided by the genome. A significant amount of the work in this area requires processing of samples which typically contain a large number of chemical compounds or molecular complexes such as proteins and peptides and complexes containing them. There is thus an ever increasing need to provide new and efficient methodologies for achieving processing of these types of samples.

In most instances, many of the processing techniques used to screen samples containing a plurality of chemical compounds, especially the processing techniques used to screen protein isolates, are based on the ability to selectively bind different target molecules or molecular complexes to a binding agent or moiety. The ability of the compounds to bind differentially is the basis of many of these techniques. For example, in micro array technology, the sample is brought into contact with the array and certain of the compounds or molecular complexes within the sample typically bind to binding moieties on the surface of the array. The unbound material is then removed (typically by washing) and the array subjected to analysis. It is thus the ability of certain compounds or molecular complexes within the sample to bind to the binding moieties on the array that provides the functionality of the array. In a similar way, many purification and/or analysis techniques require passing the sample through a column or a column-like material where the rate of transmission of material through the column is dependent upon binding interactions of the compounds or molecular complexes within the sample with the binding material in the column. Whilst in theory this should be straightforward, difficulties have been encountered especially with samples that contain proteins, especially membrane proteins.

For example, purification of chemical compounds or molecular complexes in general and proteins and peptides in particular is of significant interest in the biotechnology area predominantly due to the way that many of these compounds or molecular complexes are produced. For example, many compounds or molecular complexes of interest are produced using cell culture techniques. In these techniques a cell line is typically engineered to produce the compound or molecular complex of interest by insertion of a recombinant plasmid containing the gene coding for that compound or molecular complex. During culturing the cell lines are fed with a growth media which, although being generally tailored to meet the needs of the specific cell line, will usually contain numerous sugars, amino acids and, growth factors as well as other additives necessary or desirable to support and sustain growth. The growth media therefore contains a large number of chemical compounds other than the specific compound or compounds of interest produced by the cell line. At the end of the growth phase it is then necessary to separate the compound, protein, peptide or molecular complex of interest from the cocktail of other compounds fed to the cells during the production phase as well as from the by-products of the cells. In some instances it is also necessary to remove other compounds that are also produced by the cell in the culturing process.

Once a cell culture has been carried out, the first step in the purification process typically involves lysis of the cell which releases intracellular components into the mixture, followed by centrifugation or filtration to remove solids to produce a clear solution of a crude material to be purified containing the desired chemical compound, typically a protein, peptide or a molecular complex. The sample thus obtained is then subjected to further processing. This further processing may involve analysis, detection or purification of one or more of the components of the sample.

Current research protocols in relation to protein isolation typically involves the use of a 2D (2-dimensional) gel following, for example, a three step detergent extraction procedure. There are, however, a number of problems associated with the use of 2D gel technology. These include the expense of the procedure, poor reproducibility of the results, the fact that this process is inappropriate for low levels of protein, the fact that the sample separation is concentration dependent, the lack of performance of the procedure when applied to membrane proteins and the fact that the removal of SDS is required. It will be desirable therefore to develop alternative purification procedures for compounds or molecular complexes of this type.

A known alternative that may be used is chromatography, such as High Performance Liquid Chromatography (HPLC), Medium Pressure Liquid Chromatography (MPLC), or other known chromatographic techniques.

The chromatographic techniques utilised typically are aimed at separating the many components in the mix on the basis of either the charge, size, or hydrophobicity of the components to be separated or the affinity of the components to be separated for the particular chromatographic support used in the purification procedure.

In general, purification techniques based on chromatography rely on the fact that, with an appropriate chromatographic support, the components in the mixture can be caused to move through the chromatographic support (such as down a chromatographic column) at different rates which will be dependent upon the differences between the components in the mixture and their affinity for the chromatographic support material under the specific solvent conditions utilised. When column chromatography is used, the components are ultimately eluted from the column and, if the column conditions are appropriate, the eluted fractions, if collected in an appropriate manner, will contain predominantly the components of interest in the form of a pure compound or molecular complex. Indeed, preferably, this will provide a number of fractions containing the desired component as the only component present. To this end, in a number of chromatographic procedures such as HPLC, a detector is linked to the output of the chromatography column to detect when desired components are being eluted from the column to enable efficient collection of the fractions required.

A major class of proteins for which many of the current separation technologies have been found to be unsatisfactory, are membrane proteins which are particularly difficult to purify. It has been estimated that 30-40% of DNA codes for membrane proteins and it is anticipated therefore that this class of proteins will represent a significant proportion of the cells complement of protein. Unfortunately, over the years, the isolation of these proteins has proven to be a challenge for a number of reasons including poor solubility and conformational lability making them difficult to isolate from structurally related materials. It will be necessary for these difficulties to be overcome in order to fully utilise the opportunities provided by these proteins in the area of proteomics.

A preferred technique found to be useful for the purification of many proteins or protein complexes is HPLC which is now firmly established as a technique of choice in the analysis and purification of a wide range of chemical compounds. In this regard, HPLC has become the central technique in the characterisation of peptides and proteins and their complexes and has been crucial to many of the rapid advances made in the biological and biomedical sciences in the recent past.

The success of HPLC in protein purification can be attributed to a number of features of HPLC such as reproducibility of results, ease of selectivity, and high recovery. The most significant feature from a user's perspective is the excellent resolution obtained under a wide range of conditions for even very closely related molecules as well as structurally distinct molecules. This property of HPLC arises due to the fact that all interactive modes of chromatography are based on recognition forces which can be subtly manipulated through changes in the elution conditions that are specific for the particular mode of chromatography.

One method of improving the results obtained with all chromatographic techniques including HPLC is to modify the solid support used in the chromatographic step. In this way, modifying the solid phase for binding of the materials introduced into the column can be used to improve separation effectiveness. One challenge therefore is to provide alternative support materials that can be used in HPLC and other chromatographic purification procedures.

A number of chromatographic support materials have therefore been developed for use in general chromatography methods and for use in HPLC in particular. In preparing these materials, a number of procedures have been adopted depending on the desired support to be produced. In general, the procedures involve immobilising a binding moiety (for which the component of interest will have, or is expected to have, an affinity) on the surface of a carrier material such as silica. A number of methods have been used to immobilise such binding materials but typically this is accomplished either by covalent bonding of the binding material to the surface of the carrier in some way or by hydrophobic interaction of the binding material with the hydrophobic surface of the carrier. In some instances a combination of these two techniques is used to produce an immobilised support on the surface of the carrier.

A number of advantages and disadvantages of each system have been identified. For example, one advantage of covalent bonding of the binding moiety to the surface of the carriers is that immobilisation in this way tends to reduce leaching of the binding moiety from the surface of the carrier thus providing the chromatographic material with a longer active life when in use. In contrast, materials that rely upon hydrophobic bonding of the binding moiety to the carrier are found to have shorter life-spans as, depending on the choice of solvent, there is usually leaching of the binding moiety or binding compound from the surface of the carrier over time, rendering the chromatographic support less efficient as more and more of the binding compound is leached. Whilst this is not such a problem where the chromatographic support material is used in chromatographic techniques in which the chromatographic material is regularly replaced, such as in MPLC, it is clearly unsuitable for use in HPLC where the columns are particularly expensive and replacement of the column is undesirable and indeed uneconomic.

An advantage of the hydrophobic binding, however, is that the use of this technique means that binding material is not rigidly bound to the surface of the carrier and is more flexible. The interaction of the carrier with the binding moiety in this way, therefore, allows for greater lateral movement of the binding moiety across the surface of the carrier as required. This allows greater flexibility in potential binding of the immobilised material to compounds of interest and therefore, provides greater flexibility in forming a binding site. In addition, when one is attempting to mimic a membrane structure, it is desirable to use hydrophobic interaction as this means that the immobilised support that is produced has lateral mobility similar to the mobility observed for molecules inherently present in biological membranes. This tends to increase binding efficiency and should theoretically improve purification using these materials.

Whilst a number of proposals have been put forward to overcome or resolve these competing problems, further work is required. Thus, for example, one solution proposed to increase the “shelf life” of chromatographic materials produced by hydrophobic bonding of binding moieties to a carrier, is to cross-link at least a portion of the binding moieties which are hydrophobically bound to the surface. Typically, the crosslinking occurs at the end of the binding moiety not attached to the carrier as this is most easily cross-linked (as, for example, cross-linking is typically carried out after the binding moiety is immobilised on the column). Unfortunately, whilst this increases the shelf life of the chromatographic material, it significantly reduces mobility of the overall “surface” formed and significantly reduces the effectiveness of the material. There is therefore a need to identify binding materials that can be used in the processing of samples not only for application in purification of samples but also for application in other processing steps such as separating the components of the sample, analysis of a sample and detection of a component in a sample.

We have now found that certain binding materials have properties that can be utilised that overcome a number of these issues. These binding materials can be utilised as binding materials in micro scale chip based analysis and/or detection/analytical techniques as well as being applicable to large scale batch processing wherein the binding material is used as a chromatographic material. These binding materials are useful in the processing of samples containing compounds and molecular complexes and can be used in detection, analysis, separation, and purification of components of interest from multi component mixtures especially components of interest from protein containing samples.

The applicants have thus found that useful binding materials can be produced by incorporation of a linker of an appropriate length having certain structural features that links the binding terminal moiety to the solid support. The advantage of the use of a linker is that as the binding terminal moiety is displaced some distance from the support, the binding material, as a whole, therefore has good flexibility and conformational freedom. Where the binding moiety is based on the binding moiety of a constituent of a natural membrane, this allows the binding material to act as a membrane like structure. The supports are therefore able to have sufficient flexibility to provide good binding properties as they mimic the natural membrane surface. Furthermore, as they are covalently bonded to the support, they are able to resist leaching. In addition, a further advantage of the linkers used in the invention is that they are able to be cross-linked close to the surface of the support thus providing further strength to the binding material without compromising flexibility.

### SUMMARY OF THE INVENTION

In one aspect the present invention provides a method of separating a compound or molecular complex on the basis of the ability of the compound or molecular complex to associate with a binding material, from compounds or molecular complexes having different association characteristics, said method comprising:
(a) bringing a sample containing said compound or molecular complex into contact with a binding material, the binding material comprising:
(i) a support,
(ii) at least one terminal moiety, and
(iii) at least one linker of the formula 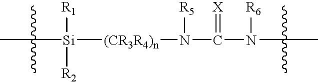
wherein R₁ and R₂ may be the same or different and are independently selected from the group consisting of: H, OH, C₁-C₆ alkyl, C₂-C₆ alkenyl, halogen, C₁-C₆ alkoxy, C₂-C₆ alkenyloxy and aryloxy, or R₁ or R₂ when taken together with an R₁ or R₂ on an adjacent linker forms a group of formula —O—, wherein said group —O— connects the silicon atom of the linker to the silicon atom of the adjacent linker,
R₃ and R₄ may be the same or different and are independently selected from the group consisting of H, C₁-C₆ alkyl, C₂-C₆ alkenyl and halogen,
R₅ is H or C₁-C₆ alkyl,
R₆ is H, C₁-C₆ alkyl, C₂-C₆ alkenyl, aryl and heteroaryl,
X is O or S,
n is an integer from 0 to 10,
   the terminal moiety being bound to the support via at least one linker,
(b) treating the product of step (a) to separate the components of the sample on the basis of their ability to associate with the binding material.

As with all chemical materials, there are preferred variables for all the substituents found in the linker. For example, it is preferred that at least one of R₁ or R₂ when taken together with an R₁ or R₂ of an adjacent linker, forms a group of formula —O—. It is particularly preferred that both R₁ and R₂ form groups of formula —O— with R₁ or R₂ on adjacent linkers, in which case, each linker is cross-linked to two other linkers. In circumstances where R₁ and R₂ do not form oxygen bridges of this type, it is preferred that they are independently selected from the group consisting of H, OH, and alkoxy. Examples of alkoxy include methoxy, ethoxy, propoxy, isopropoxy, and butoxy with ethoxy being particularly preferred,
n is preferably from 1 to 6, more preferably from 2 to 5, most preferably 3,
R₃ and R₄ are preferably selected from the group consisting of H, C₁-C₄ alkyl and halogen with H being particularly preferred,
R₅ is preferably H,
R₆ is preferably H or C₁-C₄ alkyl with H being particularly preferred,
X is preferably S.

In this context, separation of the component is intended to mean that those components with similar abilities to bind to the binding material are separated from components with a dissimilar ability to associate with the binding material. Thus, for example, if a sample has three components with two having similar association abilities with the binding material and one having a different ability to associate, the process will lead to separation of the first two components from the third. It is not intended to mean that the first two components will be separated from each other.

The terminal moiety can take a number of forms and, in principle, the terminal moiety can be any compound that has an affinity for other compounds or molecular complexes. It is preferred that the terminal moiety is selected from the group consisting of lecithins, lysolecithins, cephalins, sphingomyelin, cardiolipin, glycolipids, gangliosides, cerebrosides and phospholipids. In a particular preferred embodiment, each of the terminal moieties is independently selected from the group consisting of phosphatidylcholine, phosphatidylethanolamine, N-methyl phosphatidylethanolamine, N,N-dimethylphosphatidylethanolamine, phosphatidic acid, phosphatidylserine, phosphatidylinositol and phosphatidylglycerol or derivatives thereof.

Whilst in principle each of the terminal moieties may be bound through only one linker, it is preferred that each terminal moiety is bound to the support through two linkers. This has the advantage that it ensures that even if one binding linkage is cleaved for any reason, the terminal moiety will remain bound and leaching will not occur.

A number of supports may be utilised. It is preferred, however, that the support is selected from the group consisting of silica, alumina, titania, zirconia, polymeric resins and mixtures thereof.

It is preferred that the density of linkers on the support is greater than 1.0 mmol/m², more preferably greater than 1.8 mmol/m².

In a further aspect the present invention provides a method of detection of a compound or molecular complex in a sample containing said compound or molecular complex, said method comprising:
(a) bringing a sample containing said compound or molecular complex into contact with a binding material, the binding material comprising:
(i) a support;
(ii) at least one terminal moiety; and
(iii) at least one linker of the formula 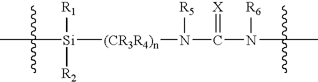
wherein R₁, R₂ may be the same or different and are independently selected from the group consisting of: H, OH, C₁-C₆ alkyl, C₂-C₆ alkenyl, halogen, C₁-C₆ alkoxy, C₂-C₈ alkenyloxy and aryloxy, or R₁ or R₂ when taken together with an R₁ or R₂ on an adjacent linker forms a group of formula —O—, wherein said group —O— connects the silicon atom of the linker to the silicon atom of the adjacent linker,
R₃ and R₄ may be the same or different and are independently selected from the group consisting of H, C₁-C₆ alkyl, C₂-C₆ alkenyl and halogen,
R₅ is H or C₁-C₆ alkyl
R₆ is H, C₁-C₆ alkyl, C₂-C₆ alkenyl, aryl, and heteroaryl,
X is O or S,
n is an integer from 0 to 10;
   the terminal moiety being attached to the support by at least one linker.

In this aspect of the invention, the linker in the binding material has the same preferred variables as discussed previously. In the methods of detection, it is preferred that the support is the solid support of an array. In the preferred embodiment in the invention, the binding material is bought into contact with the sample to allow binding of the material with the components of the sample to be detected to occur. Once the appropriate length of time has elapsed to allow the binding interactions to have occurred, the material is treated to determine the presence of the compound or molecular complex. Prior to the treatment, it is preferred that the contacted binding material is washed to remove unbound material from the sample from the binding material. The process preferably includes a step where the contacted binding material is contacted with a tagged compound which selectively binds to the compound or molecular complexes of interest that are bound to the binding material. This contacting step is then typically followed by the washing of the material to remove any unbound tagged compounds. A number of tags may be used in the tagged compounds, however, it is preferred that the tag is a radioactive tag, a fluorescent tag, or a chemiluminescent tag. The detection then typically involves analysing the binding material for the presence of the tag. The analysis may be qualitative or quantitative depending on the type of tag used and the desired result.

In an even further aspect the present invention provides a method of increasing the purity of a compound or molecular complex from a sample containing said compound or molecular complex, the method comprising:
(a) bringing said sample into contact with a binding material, the binding material comprising:
(i) a support;
(ii) at least one terminal moiety; and
(iii) at least one linker of the formula 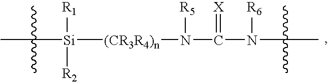
wherein R₁, R₂ may be the same or different and are independently selected from the group consisting of: —H, C₁-C₆ alkyl, C₂-C₆ alkenyl, halogen, C₁-C₆ alkoxy, C₂-C₆ alkenoxy and aryloxy, or at least one of R₁ or R₂ when taken together with R₁ or R₂ on an adjacent linker forms a group —O—, wherein said group —O— connects the silicon atom of the linker to the silicon atom of the adjacent linker,
R₃ and R₄ may be the same or different and are independently selected from the group consisting of H, C₁-C₆ alkyl, C₂-C₆ alkenyl and halogen,
R₅ is H or C₁-C₆ alkyl
R₆ is H, C₁-C₆ alkyl, C₂-C₆ alkenyl, aryl, and heteroaryl,
X is O or S,
n is an integer from 0 to 10;
   said terminal moiety being attached to said support by at least one linker;
(b) treating the product of step (a) to separate the compound or molecular complex from at least a portion of the other components of the sample; and (c) recovering at least a portion of the compound or molecular complex.

In the processes of purification of the invention, the binding material has the preferred features as discussed previously.

In one preferred embodiment, the binding material is a chromatographic material located within a chromatographic column and step (b) comprises eluting the column with a mobile phase followed by collection of eluted fractions. It is preferred that the mobile phase is selected from the group consisting of water, hydrocarbons, esters, alcohol esters, nitriles, alcohols, acids, aqueous solutions of acids and mixtures thereof with water, ethylacetate, acetylnitrile, ethanol, methanol, and aqueous solutions of trifluoroacetic acid being particularly preferred.

It is particularly preferred that the recovery step comprises combination of eluted fractions containing the same component followed by removal of the mobile phase to produce the purified compound or molecular complex. In this embodiment, the recovery step typically involves testing of the eluted fractions to determine those containing the compound or molecular complex.

An alternative embodiment utilises absorption type chromatography where the compound or molecular complex of interest is bound to the binding material in the contacting step. In this embodiment step (b) preferably comprises washing the contacted binding material to remove any unbound material. The compound or molecular complex of interest is thus increased in purity by being retained on the binding material with other compounds or molecular complexes present in the sample being removed by washing. In this embodiment, the recovery step comprises treating the contacted binding material with an agent to remove the compound or molecular complex from the binding material. Once it is removed, it is typically isolated by means well known in the art.

In yet an even further aspect the present invention provides a method of analysis of a sample containing a plurality of compounds or molecular complexes, the method comprising:
(a) bringing the sample into contact with a binding material, the binding material comprising:
(i) a support;
(ii) at least one terminal moiety; and
(iii) at least one linker of the formula 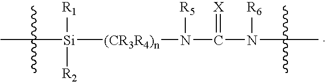
wherein R₁, R₂ may be the same or different and are independently selected from the group consisting of —H, C₁-C₆ alkyl, C₂-C₆ alkenyl, halogen, C₁-C₆ alkoxy, C₂-C₆ alkenoxy and aryloxy, or at least one of R₁ or R₂ when taken together with R₁ or R₂ on an adjacent linker forms a group —O—, wherein said group —O— connects the silicon atom of the linker to the silicon atom of the adjacent linker,
R₃ and R₄ may be the same or different and are independently selected from the group consisting of H, C₁-C₆ alkyl, C₂-C₆ alkenyl and halogen,
R₅ is H or C₁-C₆ alkyl
R₆ is H, C₁-C₆ alkyl, C₂-C₆ alkenyl, aryl, and heteroaryl,
X is O or S,
n is an integer from 0 to 10;
   said terminal moiety being attached to said support by at least one linker;
(b) treating the product of step (a) to separate at least a portion of the components of the sample; and
(c) analysing the separated components.

In the processes of analysis of the invention, the binding material has the preferred features discussed previously.

In this embodiment of the invention, the compounds or molecular complexes are preferably treated in the manner described for the purification embodiment discussed above.

In this embodiment, the method of analysis utilised in step (c) will depend on the method of separation. Thus, for example, where step (b) involves chromatographic treatment, it is preferred that the eluant from the chromatographic column is passed directly to an analytical detector such as a spectrophotometric detector or to a gas chromatograph/MS to provide mass spectral data on the eluted compounds.

Of course, in an alternative embodiment, like fractions can be combined, the component isolated and then subjected to any routine analytical technique well known in the art in order to provide a full analysis of the compounds or molecular complexes in the sample.

The present invention also relates to the use of a binding material comprising:
(i) a support;
(ii) at least one terminal moiety; and
(iii) at least one linker of the formula 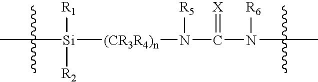
wherein R₁, R₂ may be the same or different and are independently selected from the group consisting of —H, C₁-C₆ alkyl, C₂-C₆ alkenyl, halogen, C₁-C₆ alkoxy, C₂-C₆ alkenoxy and aryloxy, or at least one of R₁ or R₂ when taken together with R₁ or R₂ on an adjacent linker forms a group —O—, wherein said group —O— connects the silicon atom of the linker to the silicon atom of the adjacent linker,
R₃ and R₄ may be the same or different and are independently selected from the group consisting of H, C₁-C₆ alkyl, C₂-C₆ alkenyl and halogen,
R₅ is H or C₁-C₆ alkyl
R₆ is H, C₁-C₆ alkyl, C₂-C₆ alkenyl, aryl, and heteroaryl,
X is O or S,
n is an integer from 0 to 10;
   said terminal moiety being attached to said support by at least one linker;
   in the analysis of a sample containing a plurality of compounds or molecular complexes.

In a further aspect, the invention relates to the use of a binding material comprising:
(i) a support;
(ii) at least one terminal moiety; and
(iii) at least one linker of the formula 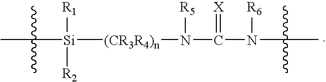
wherein R₁, R₂ may be the same or different and are independently selected from the group consisting of —H, C₁-C₆ alkyl, C₂-C₆ alkenyl, halogen, C₁-C₆ alkoxy, C₂-C₆ alkenoxy and aryloxy, or at least one of R₁ or R₂ when taken together with R₁ or R₂ on an adjacent linker forms a group —O—, wherein said group —O— connects the silicon atom of the linker to the silicon atom of the adjacent linker,
R₃ and R₄ may be the same or different and are independently selected from the group consisting of H, C₁-C₆ alkyl, C₂-C₆ alkenyl and halogen,
R₅ is H or C₁-C₆ alkyl
R₆ is H, C₁-C₆ alkyl, C₂-C₆ alkenyl, aryl, and heteroaryl,
X is O or S,
n is an integer from 0 to 10;
   said terminal moiety being attached to said support by at least one linker;
   in a method of separation or increasing the purity of a compound or molecular complex from a sample containing the compound or molecular complex.

In a final aspect, the present invention relates to the use of a binding material comprising:
(i) a support;
(ii) at least one terminal moiety; and
(iii) at least one linker of the formula 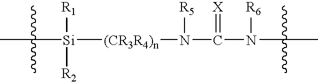
wherein R₁, R₂ may be the same or different and are independently selected from the group consisting of —H, C₁-C₆ alkyl, C₂-C₆ alkenyl, halogen, C₁-C₆ alkoxy, C₂-C₆ alkenoxy and aryloxy, or at least one of R₁ or R₂ when taken together with R₁ or R₂ on an adjacent linker forms a group —O—, wherein said group —O— connects the silicon atom of the linker to the silicon atom of the adjacent linker,
R₃ and R₄ may be the same or different and are independently selected from the group consisting of H, C₁-C₆ alkyl, C₂-C₆ alkenyl and halogen,
R₅ is H or C₁-C₆ alkyl
R₆ is H, C₁-C₆ alkyl, C₂-C₆ alkenyl, aryl, and heteroaryl,
X is O or S,
n is an integer from 0 to 10;
   said terminal moiety being attached to said support by at least one linker;
   in the detection of a compound or molecular complex in a sample.

### BRIEF DESCRIPTION OF THE DRAWINGS

FIG. 1 illustrates the structure of one of the preferred chromatographic support materials for use in the present invention, namely a phosphatidylcholine capped support material.

FIG. 2 illustrates the structure of a second preferred chromatographic support material for use in the present invention in which the terminal moiety is phosphatidic acid.

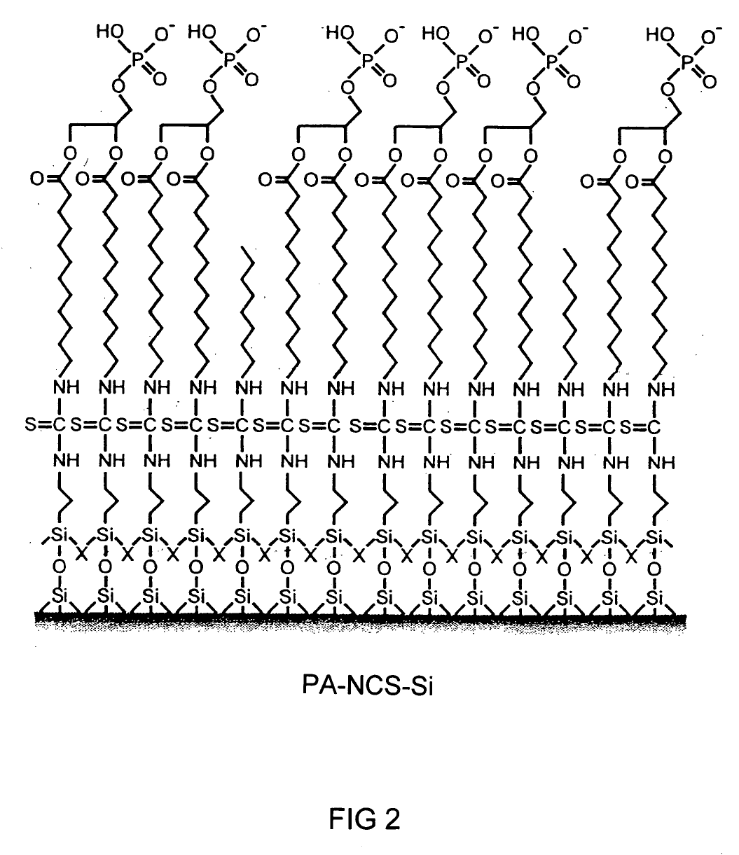

FIG. 3 illustrates yet a further preferred chromatographic support material for use in the present invention, namely immobilised phosphatidylglycerol.

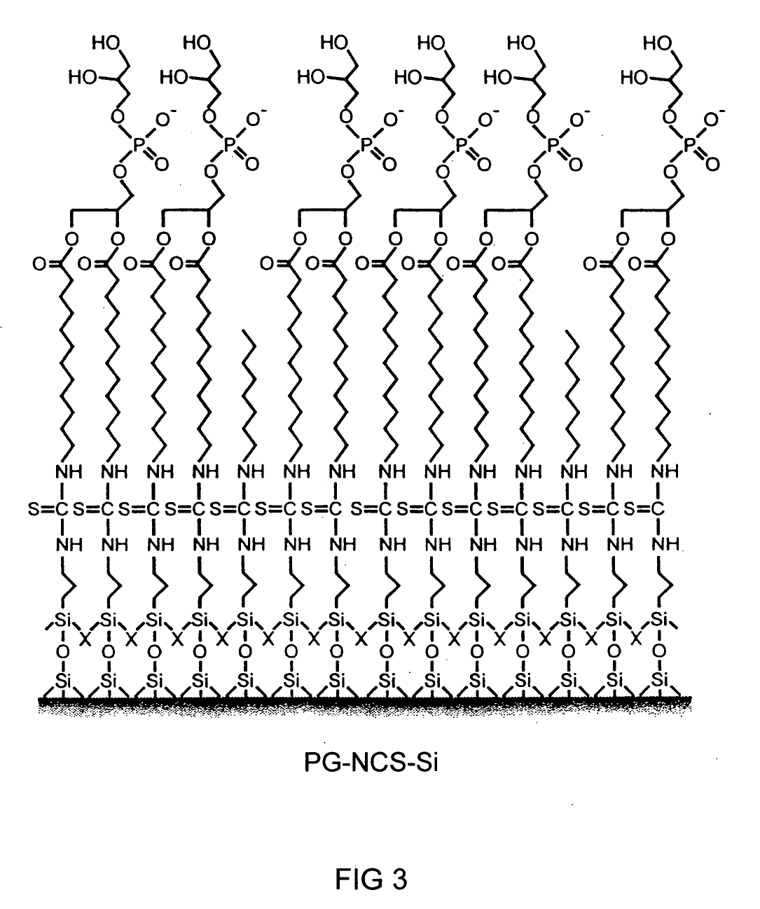

FIG. 4. This is a HPLC chromatogram of the output from example 7.

FIG. 5. This is a HPLC chromatogram of the output for example 8.

FIG. 6. This is a HPLC chromatogram for the output for example 9.

FIG. 7. This is a HPLC chromatogram for the output for example 10.

FIG. 8. This is a HPLC chromatogram for the output for example 11.

FIG. 9. This is a HPLC chromatogram for the output for example 12.

### DETAILED DESCRIPTION OF THE INVENTION

As described above, the processes of the present invention all utilise a binding material in some form. It is the ability of this binding material to differentially bind to different compounds or molecular complexes that allows the processes to achieve the desired results.

Binding Materials

The binding materials referred to and utilised in the methods of the current application are produced by covalent bonding of a terminal moiety to a support through a linker where the linker is covalently bonded both to the surface of the support and a portion of the terminal moiety. In general, a plurality of linkers are attached to the surface of the support with a plurality of terminal moieties attached to the plurality of linkers forming a membrane like structure once the terminal moieties have been attached.

The supports used as the basis for the binding materials are materials that are well known in the art. If the binding material is used in an array, the support can be any material typically used as an array support such as glass, membrane, microliter wells, mass spectrometer plates, beads or other particules, although it is preferred that it is a glass or polymeric article. If the binding material is used as a chromatographic support material, the support may be porous or non-porous and may preferably be formed of silica, alumina, titania, zirconia, polymeric resins or mixtures thereof. In general, however, it is preferred that the support materials are “pure” (in that they are made from a single material) as the methods of the present are found to be more effective if a single type of support is used. It is preferred that the support materials utilised are strong enough to withstand elevated pressures including those pressures typically found in HPLC systems, although this need not be the case where low pressure applications are desired or where the binding material is used in detection or array technology. The type of support to be utilised will be able to be determined by a skilled addressee on the basis of the particular application envisaged by the user for the binding material.

When the binding material is a chromatographic material, the support will generally be a particulate carrier material. The particle size of the carrier material can vary greatly with particles preferably having an average particle size of from about 1 micron to about 500 microns, more preferably from about 5 microns to about 100 microns, even more preferably from about 5 microns to about 50 microns, most preferably about 5 microns in diameter. It is also preferred that the particle size is relatively homogeneous as this is found to provide better resolution in the methods of the invention. The particles are also preferably porous and preferably have a pore size of the order of 300 angstroms. The preferred carrier is silica. A particularly preferred silica for use as the carrier is ZORBAX™ silica, particularly Rₓ-Sil 5 μm in diameter and 300 angstrom pore size available from Agilent Technologies. In general, these materials are generally commercially available.

A number of terminal moieties can be used in the binding material used in the methods of the invention with the particular terminal moiety chosen depending on the chemical compound or molecular complex desired to be subjected to the process. In principle, however, any chemical moiety can act as the terminal moiety as all chemical compounds have at least some binding affinity to at least one other chemical compound or molecular complex. The role of the terminal moiety is to provide a binding interaction with at least one of the compounds in the sample. A large number of species can therefore be used as the binding moiety and a skilled addressee will generally know which type of terminal moiety to use to achieve the desired binding interaction. Merely by way of example, if the application is an array it is preferred that the terminal moiety is a ligand binding reagent such as an antibody.

When the process is increasing the purity of a compound or molecular complex in a sample using the binding material as a chromatographic material, for example, the terminal moiety is typically chosen so that it has some binding affinity with the compound of interest sufficient to provide some increase in purity but which does not bind too strongly to the material to be isolated. If the material binds too strongly, the compound or molecular complex will not be able to be removed from the chromatographic material rendering the process far more difficult. In general, a skilled worker will find little difficulty in determining whether the binding of the terminal moiety to the compounds of interest is sufficient to provide some purification and/or if the binding is too strong.

When the process is detection, however, it is desirable that the binding moiety binds strongly (and sometime even irreversibly) to the binding moiety. Such strong binding allows the sample to be bought into contact with the binding material in a manner whereby the compound or molecular complex of interest binds to the binding material. The binding material is then treated with material to remove unbound material (typically such as by washing). It is therefore important when the binding material is used in this way that the washing will not remove the bound compound or molecular complex of interest.

Preferred terminal moieties for incorporation in the chromatographic support material include lecithins, lysolecithins, cephalins, sphingomyelin, cardiolipin, glycolipids, gangliosides, cerebrosides and phospholipids or derivatives thereof. These are found to be particularly useful when the processes of the present invention are applied to proteins or peptides, particularly membrane proteins. It has been found that as these materials are commonly found in cell membranes, they have an affinity for membrane proteins and thus are very useful when samples containing membrane proteins are subjected to the processes described herein.

Of these, it is particularly preferred that the terminal moieties are phospholipids and derivatives thereof. The phospholipid is preferably selected from the group consisting of phosphatidylcholine, phosphatidylethanolamine, N-methyl phospatidylethanolamine, N,N-dimethylphosphatidylethanolamine, phosphatidic acid, phosphatidylserine, phosphatidylinositol and phosphatidylglycerol or derivatives thereof. Most preferably, the terminal moiety is selected from the group consisting of phosphatidic acid, phosphatidylglycerol, phosphatidylcholine or derivatives thereof.

The general structural formula of many of these terminal binding moieties is as follows:

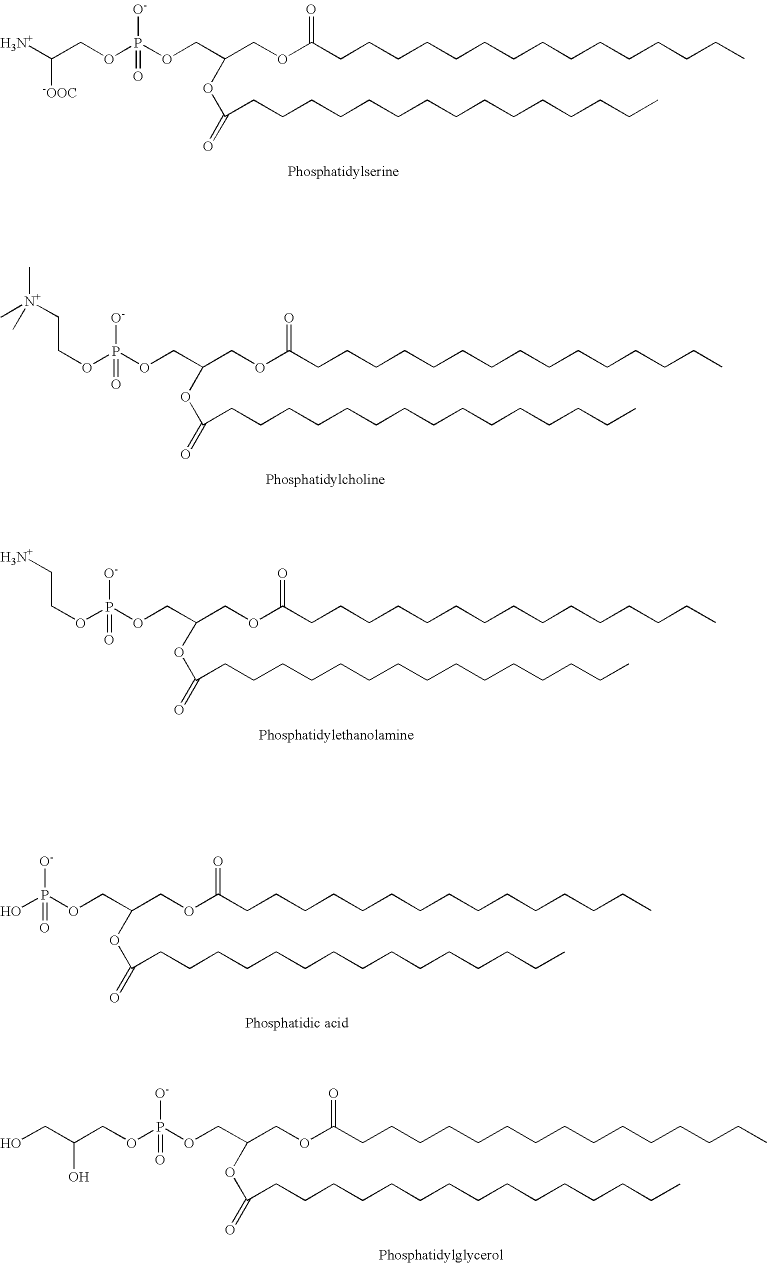

As can be seen, these materials have two aliphatic chains. When they are used as terminal moieties, they are typically bound to the linker via the terminal carbon of at least one of the aliphatic chains. Preferably, each terminal moiety is bound to two linkers with each aliphatic chain being attached to a linker. In this way, if the contact with the linker is cleaved in one chain, the other chain holds the moiety bound to the support. Derivatives of the natural materials may also be used in which the length of the aliphatic chains are varied. When this occurs it is preferred that the aliphatic chains contain the same number of carbons. The number of carbons in the aliphatic chains in these variants is from 1 to 20, more preferably 10 to 16, most preferably 11 carbons. In addition, derivatives of the natural materials may also be achieved by introduction of elements of unsaturation into the carbon backbone. One advantage of introduction of unsaturation into the carbon chain is that it can provide additional functionality for further elaboration of the terminal moiety. Alternatively, once the terminal moiety is bound, the presence of unsaturation allows the further cross-linking of the membrane if required.

Of course, as would be clear to a skilled addressee, the carbon chains may also be substituted with any suitable non-interfering substituent. A non-interfering substituent is any substituent that does not interfere with the binding of the terminal moiety with the linker.

The terminal moieties discussed above are typically immobilised to the support material via one or more linkers. Preferably each terminal moiety is attached to the support through two linkers. The linkers used in the binding materials used in this application have the following formula 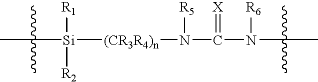
wherein R₁, R₂ may be the same or different and are independently selected from the group consisting of —H, C₁-C₆ alkyl, C₂-C₆ alkenyl, halogen, C₁-C₆ alkoxy, C₂-C₆ alkenoxy, aryloxy, or R₁ or R₂ together with R₁ or R₂ on an adjacent linker forms a group —O— wherein said group connects the silicon atom of the linker to the silicon atom of the adjacent linker,
R₃ and R₄ may be the same or different and are independently selected from the group consisting of H, C₁-C₆ alkyl, C₂-C₆ alkenyl and halogen,
R₅ is H or C₁-C₆ alkyl
R₆ is H, C₁-C₆ alkyl, C₂-C₆ alkenyl, aryl, and heteroaryl;
X is O or S;
n is an integer from 0 to 10.

It is preferred that at least one of, and most preferably both of R₁ and R₂ independently together with an R₁ and R₂ group on an adjacent linker, form a group —O— (an oxygen bridge) between the two linkers. As such, it is preferred that each linker is attached to at least one, most preferably at least two other linkers via —O— “bridges”. The provision of an oxygen bridge of this type serves to increase the structural integrity of the linkers providing added strength to the binding material ultimately produced. In effect, these bridges preferably form a 3 dimensional mesh like structure wherein the linkers on the surface of the support are interconnected by the oxygen bridges.

As with all chemical materials, there are preferred variables for all the substituents. For example, as discussed above, it is preferred that at least one of R₁ or R₂ when taken together with an R₁ or R₂ of an adjacent linker, forms a group of formula —O—. It is particularly preferred that both R₁ and R₂ form groups of formula —O— with R₁ or R₂ on adjacent linkers. In circumstances where R₁ and R₂ do not form oxygen bridges of this type, it is preferred that they are independently selected from the group consisting of H, OH, and alkoxy. Examples of alkoxy include methoxy, ethoxy, propoxy, isopropoxy, and butoxy with ethoxy being particularly preferred,
n is preferably from 1 to 6, more preferably from 2 to 5, most preferably 3,
R₃ and R₄ are preferably selected from the group consisting of H, C₁-C₄ alkyl and halogen with H being particularly preferred,
R₅ is preferably H,
R₆ is preferably H or C₁-C₄ alkyl with H being particularly preferred,
X is preferably S.

A particularly preferred linker is of formula:

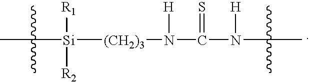

It is preferred that the Silicon terminus of the linker is attached to the support material with the nitrogen terminus being preferably bound to the terminal moiety.

It is preferred that there are a plurality of linkers attached to the support material with a plurality of terminal moieties attached to the plurality of linkers. It is preferred that the density of linkers on the support material is of greater than 1.0 mmol/m², preferably greater than 1.8 mmol/m².

The synthesis of the binding material may be carried out in a number of ways. It is typical, however, that a linker precursor is first attached to the support followed by reaction of a suitably activated terminal moiety with the bound linker precursor to produce the final binding material. In some instances, in order to ensure that any unreacted linker precursor does not interfere, an end-capping step is conducted in which the unreacted linker precursor are reacted with a blocking moiety such as octylamine.

The first step in the synthesis involves activation of the support. This may be carried out in any way known in the art and preferably produces an activated support having free hydroxyl groups on the surface of the support. The activated support is then typically reacted with a linker precursor of the formula 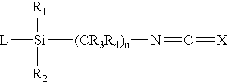
wherein L is a leaving group, and R₁, R₂, R₃, R₄, n and X are as previously defined to produce a linker precursor bound to the surface of the support. This is then reacted with an amino substituted (preferably a di-amino substituted) terminal moiety to produce the bound terminal moiety. Where the di-amino derivative is used, the terminal moiety is bound via two linkers.

The synthesis of three preferred terminal moieties and their linking to supports to make the chromatographic support materials will now be discussed.

Synthesis of the Phosphatidylcholine Material for Immobilisation

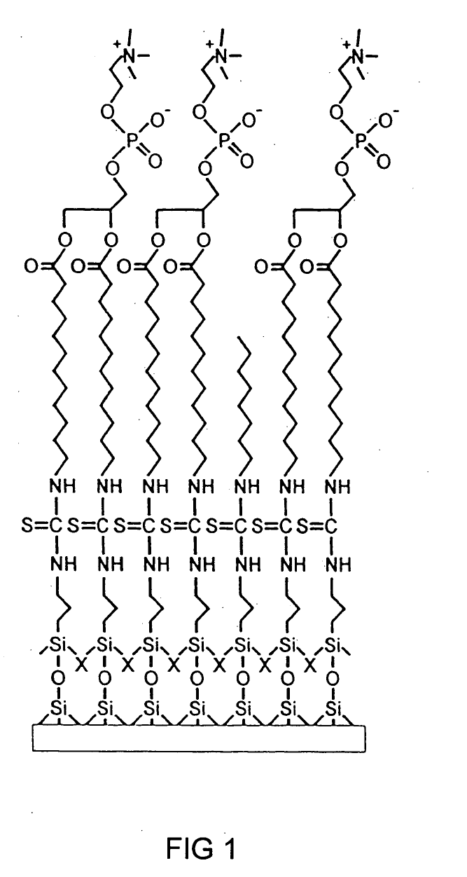

The phosphatidylcholine modified silica material as shown in FIG. 1 was produced by coupling a modified phosphatidylcholine derivative to a modified silica. The synthesis of the derivative for coupling with the modified silica is shown in scheme 1. The first step involves reaction of commercially available N-(benzyloxycarbonyloxy)-succinimide with commercially available 12-amino dodecanoic acid (1) to protect the terminal amino group as the boc derivative (2) which was obtained in good overall yield. Reaction of the boc protected amino dodecanoic acid (2) with dicyclohexylcarbodiamide (DCC) then produced the corresponding anhydride (3) from condensation of the free acid. Reaction of the acid (3) with the cadmium chloride complex of sn-glycero-3-phosphorylcholine (GPG) in the presence of dimethylaminopyridine (DMAP) under conditions of azeotropic removal of water produced the protected phosphorylcholine derivative (4). The reaction of (4) with paladium on carbon under an atmosphere of hydrogen smoothly removes the boc protecting group producing the amine (5). This material was now ready for immobilization on the silica. Upon immobilisation of the linker to the support, it was then reacted with the amine(s) to produce the desired binding material. As can be seen, the support (silica) is bound through two linkers to the terminal phosphatidyl choline group. 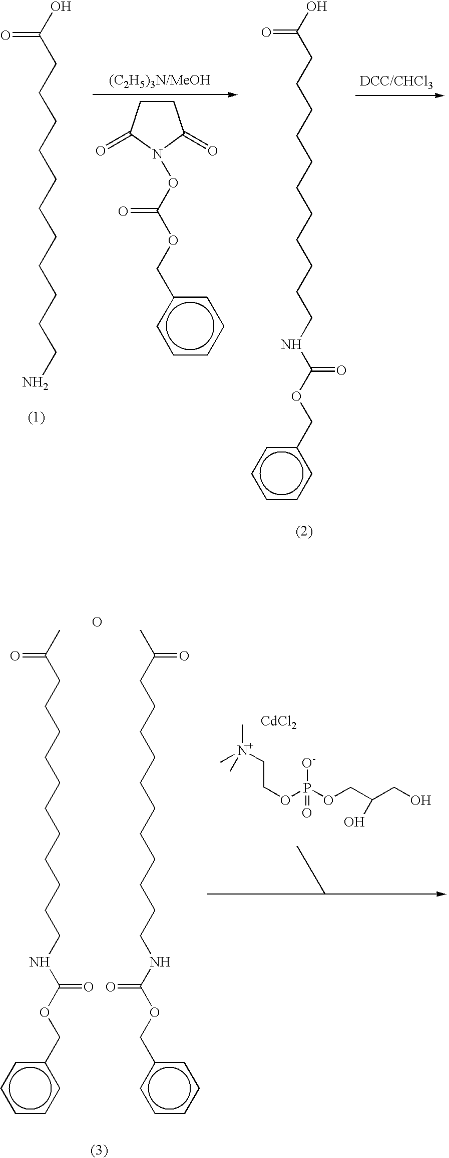 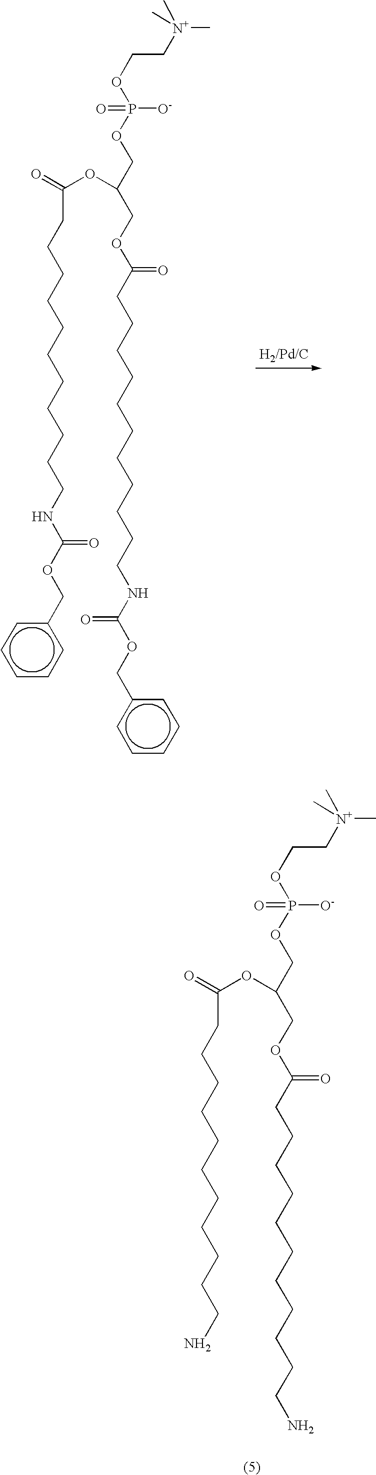
Synthesis of the Phosphatidic Acid Derivative

The synthesis of the phosphatidic acid derivative is shown in scheme 2.

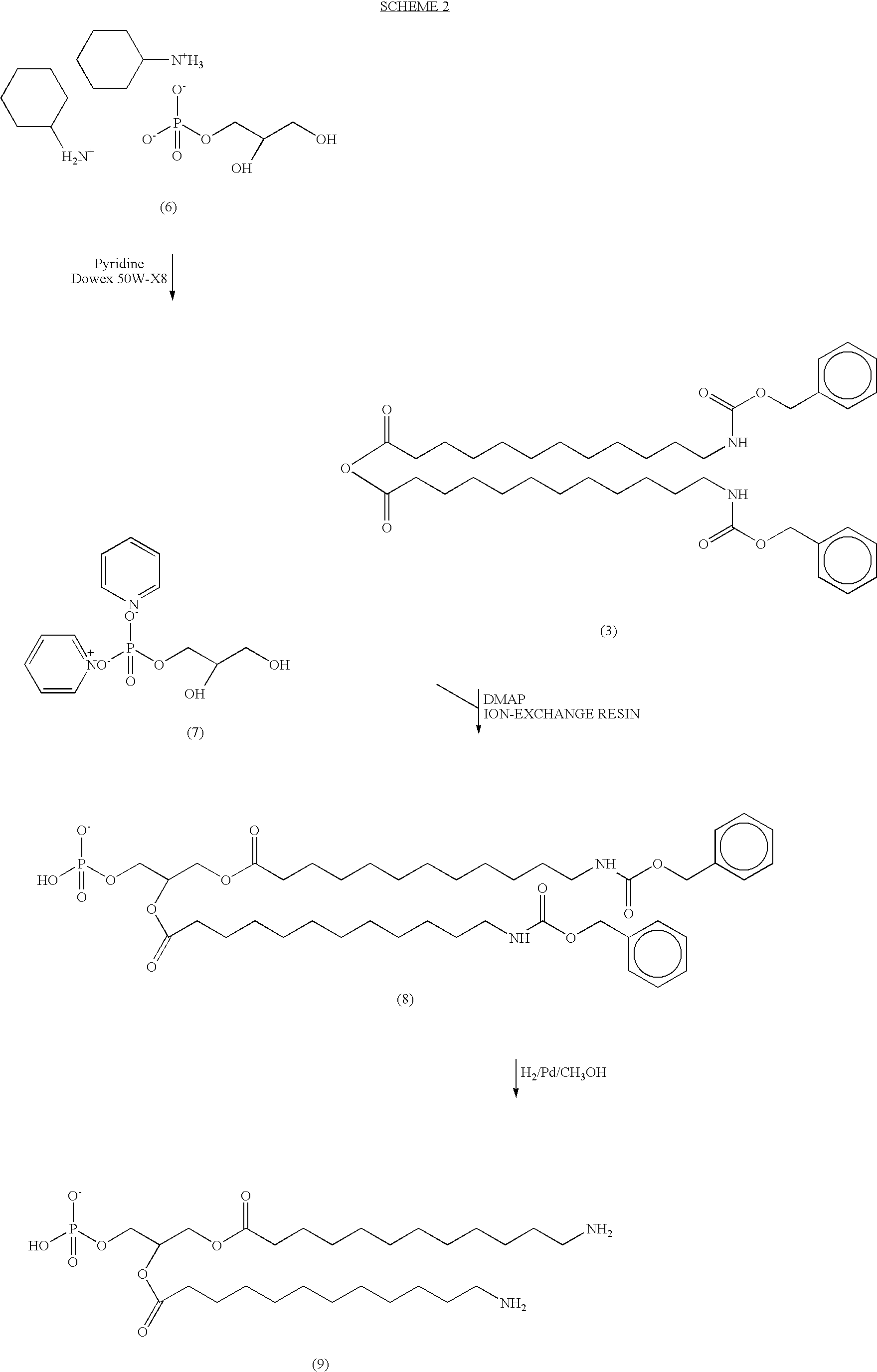

Thus, di-cyclo hexyl ammonium sn-glycero-3-phosphate (6) was converted to the pyridinium salt by passing an aqueous solution of the sn-glycero-3-phosphate (6) through pyridinium ion exchange resin to produce the pyridinium salt (7). The material (7) was not isolated but was immediately re-suspended and sonicated with three mole equivalents of dimethylaminopyridine (DMAP) and freshly prepared N-benzyloxycarbonyl-12-aminododecenoic acid-anhydride (3) to produce 1,2-di-0-N-benzyloxycarbonyl-12-aminododecenyl(—SN-glycero-3-0-phosphate) (8). This was then reacted with palladium on carbon under an atmosphere of nitrogen to produce the phosphatidic acid derivative (9). This material was then ready for coupling to the modified silica.

Synthesis of Phosphatidylglycerol Derivative

The synthesis of the phosphatidylglycerol derivative was performed as shown in scheme 3. Thus, the protected phosphatidylcholine derivative (4) was reacted with phospholipase D from Streptomyces species in the presence of L-2,3-0-isopropylidene-sn-glycerol to form the desired product 10. Deprotection of the boc protecting group by palladium on carbon then produced the amino derivative (11) which was suitable for binding to the linker. 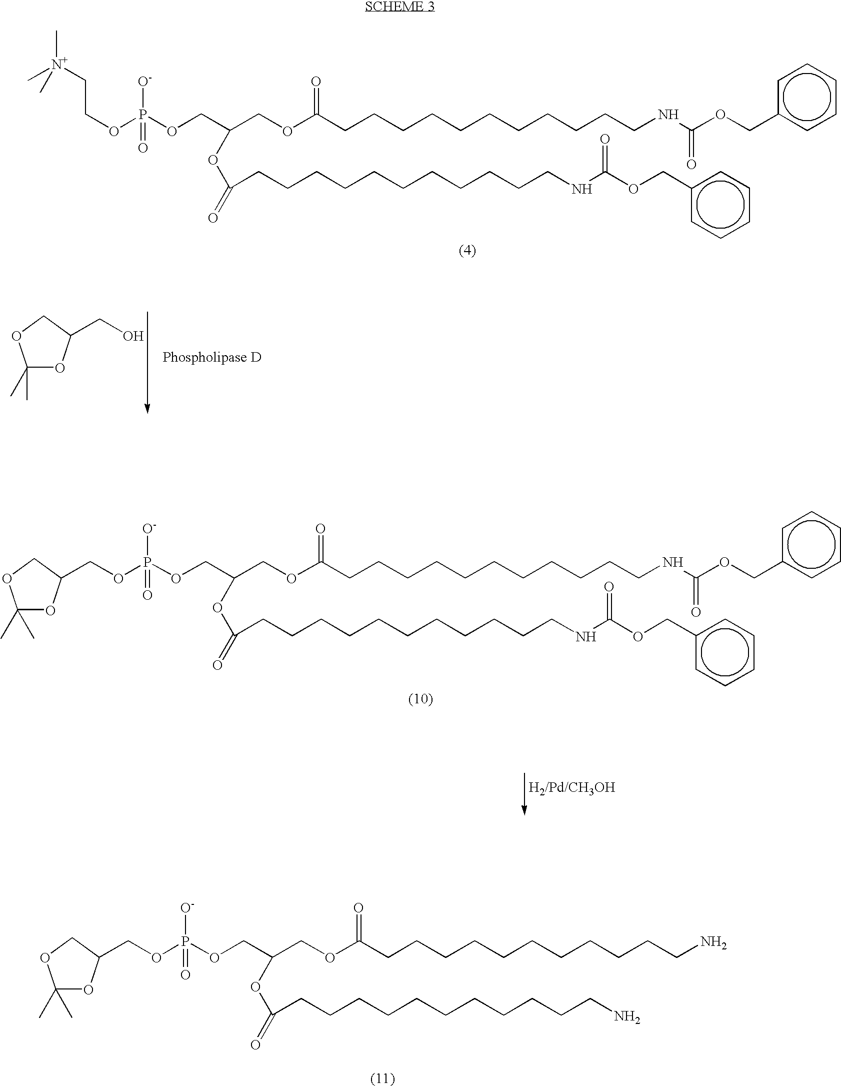
Immobilisation of the Terminal Groups

The sequential series of steps required to produce the solid support from silica are outlined in scheme 4. As will be apparent to a skilled addressee, the surface of silica has a number of hydroxy residues attached thereto as shown as (a) in scheme 4. The silica was activated by suspending the particles in 100 ml isopropanol with the addition of a few drops of hydrochloric acid to neutralise the solution and sonicated for 10 minutes.

The silica suspension thus formed was then rotary mixed at a medium speed for 24 hours. The resulting porous silica suspension was centrifuged at 2000 rpm for 10-15 minutes. After decanting the clear supernatant, the particles were then washed with water and isopropanol. The porous silica particles were then dried at 120° C. for 24 hours. Any remaining water was removed from the surface of the silica by heating it to 180° C. under vacuum. The activated silica particles thus formed which are depicted as (a) in scheme 4 were then reacted with 3-isothiocyanalopropyl triethoxy silane (ITCPS) in the presence of catalytic amounts of imidazole. This produced a modified surface shown as (b) in scheme 4. Reaction of the modified silica particles with a material to be immobilised such as compound (5) then leads to a surface as shown in (c) in scheme 4 in which a large number of the isothio groups have reacted with the phosphatidylcholine derivative. Finally, the material was reacted with octylamine under similar reacting conditions to ensure complete reaction of any unreacted isothiocyanate groups which could interfere with the surface at a later date. This then produces a surface having features outlined as (d) in scheme 4. 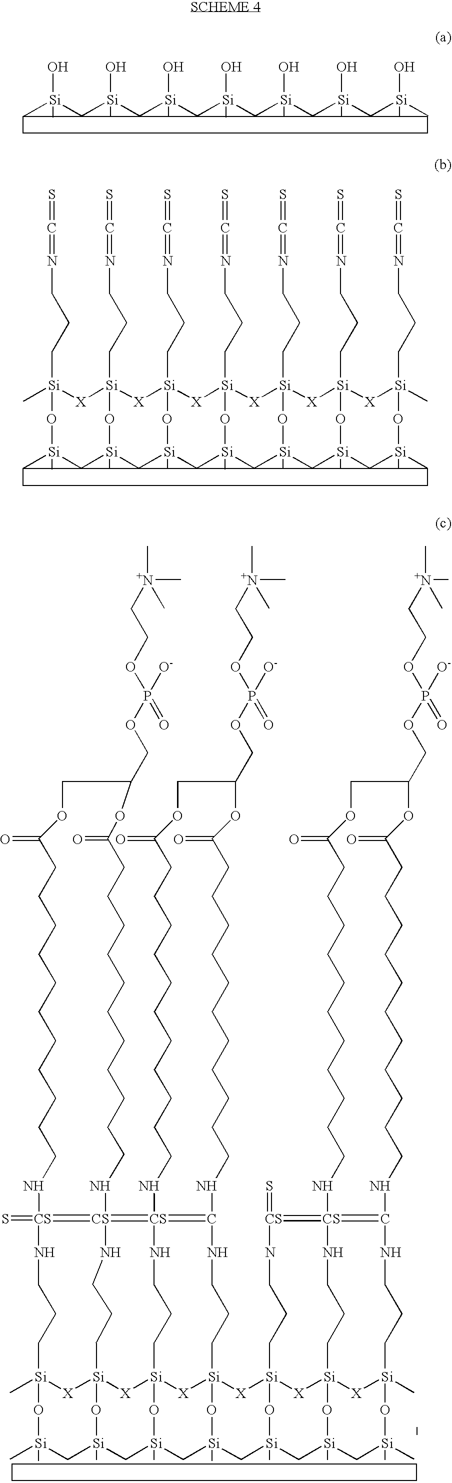 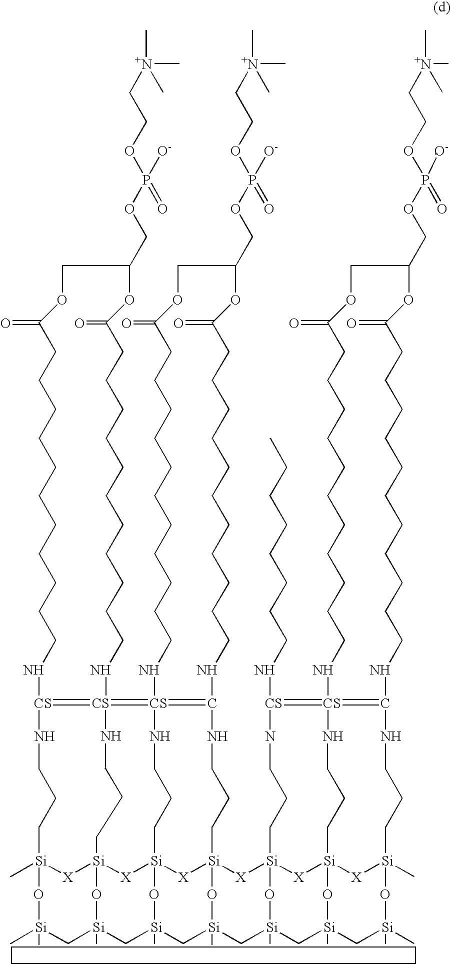
Processes of the Invention

The processes of the invention are all conducted on samples containing a compound or molecular complex of interest. Typically, these samples contain a plurality of chemical compounds, a plurality of molecular complexes or a mixture of both. The sample can be from any suitable source including from a chemical synthesis reaction or a fermentation broth. It is preferred, however, that the sample is derived from a cell or a viral homogenate. Procedures for producing samples derived from cells or viral homogenates are well known to a skilled address in the art and will not be repeated here. It is preferred that this sample contains proteins, peptides or complexes of proteins and peptides. It is particularly preferred that the proteins are membrane proteins.

Sample Preparation

In principle, the samples to be subjected to the processes of the present invention may be of any level of purity when they are subjected to the inventive processes. As would be clear to a skilled addressee, however, it is typical that samples are filtered to remove any particulate material prior to subjection to the processes of the invention. Whilst this step can be dispensed with, it is typically found that if the sample contains particulate matter and is not filtered, then the particulate will be likely to clog the binding material, thus lowering performance. It is also typical that the samples are also filtered through a small “pad” of a chromatographic material to remove “baseline” materials that bind irreversibly to the material, thus destroying the useful life of any binding material they are bought in contact with. This is particularly true where the process is purification in a column. These two pretreatment steps are preferred steps but not essential to the working of the invention.

The Processes

In one embodiment the present invention provides a method of separating a compound or molecular complex on the basis of the ability of the compound or molecular complex to associate with a binding material, from compounds or molecular complexes having different association characteristics, said method comprising:
(a) bringing a sample containing said compound or molecular complex into contact with a binding material, the binding material comprising:
(i) a support,
(ii) at least one terminal moiety, and
(iii) at least one linker of the formula 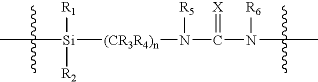
wherein R₁ and R₂ may be the same or different and are independently selected from the group consisting of: H, OH, C₁-C₆ alkyl, C₂-C₆ alkenyl, halogen, C₁-C₆ alkoxy, C₂-C₆ alkenyloxy and aryloxy, or R₁ or R₂ when taken together with an R₁ or R₂ on an adjacent linker forms a group of formula —O—, wherein said group —O— connects the silicon atom of the linker to the silicon atom of the adjacent linker,
R₃ and R₄ may be the same or different and are independently selected from the group consisting of H, C₁-C₆ alkyl, C₂-C₆ alkenyl and halogen,
R₅ is H or C₁-C₆ alkyl,
R₆ is H, C₁-C₆ alkyl, C₂-C₆ alkenyl, aryl and heteroaryl,
X is O or S,
n is an integer from 0 to 10,
   the terminal moiety being bound to the support via at least one linker,
(b) treating the product of step (a) to separate the components of the sample on the basis of their ability to associate with the binding material.

In a further embodiment, the present invention provides a method of increasing the purity of a compound or molecular complex from an impure sample containing said compound or molecular complex, the method comprising:
(a) bringing said sample into contact with a binding material, the binding material comprising:
(i) a support;
(ii) at least one terminal moiety; and
(iii) at least one linker of the formula 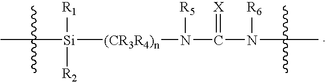
wherein R₁, R₂ may be the same or different and are independently selected from the group consisting of: —H, C₁-C₆ alkyl, C₂-C₆ alkenyl, halogen, C₁-C₆ alkoxy, C₂-C₆ alkenoxy and aryloxy, or at least one of R₁ or R₂ when taken together with R₁ or R₂ on an adjacent linker forms a group —O—, wherein said group —O— connects the silicon atom of the linker to the silicon atom of the adjacent linker,
R₃ and R₄ may be the same or different and are independently selected from the group consisting of H, C₁-C₆ alkyl, C₂-C₆ alkenyl and halogen,
R₅ is H or C₁-C₆ alkyl
R₆ is H, C₁-C₆ alkyl, C₂-C₆ alkenyl, aryl, and heteroaryl,
X is O or S,
n is an integer from 0 to 10;
   said terminal moiety being attached to said support by at least one linker;
(b) treating the product of step (a) to separate the compound or molecular complex from at least a portion of the other components in the sample;
(c) recovering at least a portion of the compound or molecular complex.

Whilst, in principle, the level of purity of the initial sample in each of these processes is irrelevant, it is typically found that the greater the initial purity of the compound or molecular complex in the sample subjected to the separation/purification increase procedure, the greater the resolution will be and the more improved the results obtained will be. Thus, it is preferred that the desired compound or molecular complex constitutes at least 10%, more preferably at least 30%, even more preferably at least 50%, even more preferably at least 70%, most preferably at least 90% of the total amount of material subjected to the separation/purification increase procedure.

The methods of the present invention are applicable to almost any chemical compound or molecular complex. The compounds or molecular complexes that can be processed effectively by the procedures of the present invention are, in principle, preferably chemical compounds or molecular complexes that have a binding affinity for the terminal moiety chosen. It is particularly preferred that the compound is an organic compound, preferably a peptide, protein, or glycoprotein. Most preferable, the protein is a membrane protein having at least one hydrophobic domain or region. If the compound is a protein, it is preferably a high molecular weight protein. These can be obtained from any source, however, it is preferred that the sample is derived from a cell or viral homogenate.

In the method of increasing the purity processes of the present invention, the binding material is preferably utilised as a chromatographic support material in chromatographic systems well known in the art. The binding material discussed previously is suitable for use in any of a number of chromatographic systems that have been utilised for purifications. Thus, they can be utilised in simple applications where the mixture is filtered through a small pad of the chromatographic support material up to and including more complex applications such as HPLC. The binding materials can therefore be utilised in traditional and well known chromatographic liquid/solid applications as well as thin layer chromatography, MPLC and in HPLC systems. The binding material can therefore be utilised in any chromatographic system known in the art.

In an alternative embodiment, the binding material may be located on a flat support or plate. In these embodiments, the sample is bought into contact with the binding material, thereby binding compounds or molecular complexes of interest. The plate is then preferably washed to remove unbound materials. Following washing (if applicable), the binding material is then treated to remove the compound or molecular complex from the support (in increased purity) or in context with compounds or molecular complexes with similar binding ability (in separation).

In essence, therefore, irrespective of the embodiment of the separation or increased purification process, the step of bringing a sample into contact with a binding material comprises adding the sample to the binding material. Where the binding material is located within a chromatographic column, this typically involves loading the sample onto the column. Where the binding material is on an assay plate such as a microarray, the contacting typically involves pipetting the sample onto the surface of the array.

It is preferred, however, that the process occurs where the binding material is a chromatographic support material in a chromatographic column. In these systems, the chromatographic support material is typically utilised in the form of a packed column such as a medium pressure liquid chromatography column or a high performance liquid chromatography column. Methods for packing columns using chromatographic support materials and columns that can be packed with these types of materials are well known in the art. In addition, column geometry is also well known. Once a column has been packed (typically by air pressure packing) into a chromatographic column, it is typically then contacted with a solvent or mobile phase to produce a “wetted” column suitable for use. It is typical that the solvent or mobile phase used for “wetting” the column will depend on the type of compounds to be purified. Typically, the solvent or mobile phase utilised is chosen such that the compound to be purified has a very low mobility on the chromatographic support when that solvent or mobile phase is utilised. (To ensure that migration of the loaded materials through the column does not occur before elution is commenced).

In the process, it is preferred that the treating step involves elution of the contacted material with a mobile phase with collection of eluted fractions. Accordingly, once the material has been loaded on the chromatographic support material, it is then typical to elute the contacted material with a mobile phase so that the compounds migrate through the column of chromatographic material and eventually are expelled from the column. Methods of elution of this type are well known.

In principle, any solvent or solvent mixture can be used as the mobile phase in the processes of the present invention although aqueous and/or organic solvents are particularly preferred. Suitable solvent and/or solvent mixtures for use in the methods of the present invention can include hydrocarbons, ethers, alkyl esters, nitriles, alcohols, and acids. Particularly preferred solvents include water, methanol, ethanol, ethylacetate, acetonitrile, and aqueous solutions of trifluoro-acetic acid. It is also typical that mixed solvent systems are used either as a single solvent or in a solvent gradient system.

The process of the present invention, typically involves changing the elution conditions of the column over time. Thus, for example, it is typical that initial elution of the column occurs with one solvent with the percentage of the second solvent in the solvent mixture added to the column gradually being increased over time, thus providing a solvent gradient. Such gradient solvent techniques are well known in the art and typically involve starting with 100% use of solvent (A) and 0% of solvent (B) and ending up at the end of the elution run with 100% solvent (B) and 0% solvent (A). The preferred first and second solvents for use in the purifications of the present invention involve solutions of trifluoroacetic acid in water (preferably 0.1% trifluoroacetic acid in water) and a mixture of 40% 0.1% TFA in water/60% acetonitrile.

Methods of altering the elution gradient in the manner described above are well known in the art and would be clear to a skilled addressee. In principle, the elution gradient can be varied with the start points and end points of the elution gradient being determined by a skilled addressee on the basis of the compound to be purified and the time restraints presented. In principle, however, any elution gradient can be chosen and the exact elution gradient to be chosen would depend on the compound to be purified. The fractions eluted from the column are then typically recovered in portions which preferably contain pure material of the compounds to be isolated. The size of the portions to be collected can range widely and the number of portions collected for each column will also vary greatly. Thus, for example, in medium pressure liquid chromatography a large number of separate portions may be collected with each portion consisting of between 1 and 5 ml with a total of between 30 and 150 separate fractions being isolated. These fractions are then typically tested for presence of the desired compound and the fractions containing the desired compound are combined and the desired compound or molecular complex recovered from the fraction.

In some techniques, detectors are placed on the outlet of the chromatographic column to detect when the desired compound or molecular complex is being eluted from the column and, hence, allow more efficient collection. There are a number of detectors well known in the art which are suitable for this purpose. The use of these detectors allows the collection of fractions composed predominantly of a compound of interest and may avoid the need for multiple fractions to be collected.

Recovery of the eluted compounds or molecular complexes from fractions that contain it may occur in a number of ways well known in the art. In circumstances where the mobile phase is purely an organic solvent, the desired compound or molecular complex is typically recovered by distilling off the solvent using conventional techniques. A rotary evaporator is typically utilised in such a step.

Alternatively, with some solvent systems it may be necessary to extract the fraction with an organic solvent under conditions necessary to partition the desired compound into the organic phase. The compound can then be recovered from the organic phase as discussed above. Other methods such as by crystallising the desired compound from the fraction can also be utilised.

It may also be necessary to repeat the process of the present invention a number of times to achieve the desired level of increased purity and/or separation levels for the compound or molecular complex of interest. In these circumstances, those fractions containing the desired compound or molecular complex are typically combined, concentrated and re-subjected to the process of the invention to provide a product of greater purity.

Preferably, after subjection to the method of the invention, the desired compound has a purity of at least 80%, more preferably 90%, even more preferably at least 95%, even more preferably at least 97%, most preferably at least 99%. It is particularly preferred that after subjection to the process, the compound is recovered in substantially pure form.

In an alternative embodiment, the binding materials may be used in capillary electrochromatography (CEC). This is a rapidly evolving hybrid technique between HPLC and capillary electrophoresis (CE). In essence, in this technique, CE capillaries are packed with a chromatographic support material (i.e., the binding material) and a voltage is applied across the packed capillary which generates electro osmotic flow (EOF). The EOF transports solutes along the capillary capillaries towards a detector. In this technique both differential partitioning based on the binding of the solute to the binding material and electrophoretic migration of the solute occurs during the transportation towards the detector leading to CEC separation of the constituents of the sample. In general, with the use of this technique, it is possible to obtain unique separation selectivities when compared to either HPLC or capillary electrophoresis alone. It is generally found that the flow profile of EOF reduces flow related band broadening and separation/purification efficiencies are generally considerably higher. In addition, carrier electrolytes that contain high levels (typically between 40-80%) of organic solvents such as methanol and acetonitrile are employed in this technique. As such, resolution of both water in soluble and neutral solutes is readily achieved. In general, this technique, as stated previously, is a hybrid of capillary electrophoresis and HPLC. This is not described in great detail in this specification as it is considered that capillary electrophoresis and even capillary electrochromatography are sufficiently well known to be understood by a skilled addressee as are the process steps involved.

The processes of separation and increasing the purity contemplated in this application and discussed above therefore contain a number of very similar steps. In general, the difference is the aim of the process. When increasing the purity, for example, the aim is to preferably provide fractions containing pure or relatively pure components from an impure sample. This means that the aim, therefore, is to ensure that the treatment step (b) is such that the compound or molecular complex is separated preferably in a pure or relatively pure form from the other components of the sample (which may or may not be purified). The aim of this is to allow characterisation of the component of interest in the sample or to provide sufficient amounts of compound or molecular complex for further elaboration. This is particularly applicable where the sample has been obtained for a process where the aim is to produce viable amounts of compound or molecular complex for further reactions.

In contrast, with separation, the aim is not necessarily to produce a compound or molecular complex in pure form (or even to increase the purity) but rather is to gain information on the components in the sample as to their ability to bind to certain binding materials. In this process, the aim is therefore to identify compounds with similar abilities to bind to the binding materials. The provision of data of this type allows a skilled worker to determine whether the components of the sample have similar affinity to the terminal moiety of the binding material. This information can be very useful as where the terminal moiety is designed to mimic a natural membrane, the separation disclosed herein allows a skilled worker to determine which compounds have similar binding affinities for the membrane and which have dissimilar binding characteristics.

Where two compounds separate together, it is concluded that they have similar binding characteristics for the binding material. The provision of information of this type thus provides insight into the sorts of material that bind to the membrane and can be helpful in structural elucidation of the binding interaction.

In particular, the separation technique can be useful in circumstances where a worker knows that a particular compound or molecular complex has affinity for a membrane. By doping the sample with the compound that has a known affinity to bind to the binding material, a skilled worker using the separation technique can determine which components in the sample separate with the compound or molecular complex with the known binding affinity. This provides the worker with information as to whether any components of the sample have similar binding characteristics to the known compound. In addition, as the ability to bind is generally inversely proportional to the speed of elution through the column, for example, qualitative information can be obtained by doping a sample with a compound in which the binding affinity is known.

The separation process described herein therefore allows a skilled worker to probe interactions of the components of the sample with the particular binding moiety chosen. Thus, for example, where the binding moiety is chosen, such that the binding material mimics a natural membrane, the separation technique can be used to probe interactions of the components of the samples with the membrane. In addition, as the separation technique is a relatively soft technique and does not harm protein/protein interactions, it allows analysis by the skilled worker of protein/protein interactions in the original sample. Accordingly, where there are protein/protein interactions in the sample, the separation technique eluded to allows for identification of this (as the two proteins will separate together).

In a further aspect the present invention provides a method of detection of a compound or molecular complex in a sample containing said compound or molecular complex, said method comprising:
(a) bringing a sample containing said compound or molecular complex into contact with a binding material, the binding material comprising:
(i) a support;
(ii) at least one terminal moiety; and
(iii) at least one linker of the formula 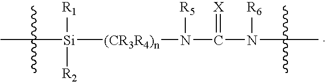
   wherein R₁, R₂ may be the same or different and are independently selected from the group consisting of: H, OH, C₁-C₆ alky, C₂-C₆ alkenyl, halogen, C₁-C₆ alkoxy, C₂-C₆ alkenyloxy and aryloxy, or R₁ or R₂ when taken together with an R₁ or R₂ on an adjacent linker forms a group of formula —O—, wherein said group —O— connects the silicon atom of the linker to the silicon atom of the adjacent linker,
R₃ and R₄ may be the same or different and are independently selected from the group consisting of H, C₁-C₆ alkyl, C₂-C₆ alkenyl and halogen,
R₅ is H or C₁-C₆ alkyl
R₆ is H, C₁-C₆ alkyl, C₂-C₆ alkenyl, aryl, and heteroaryl,
X is O or S,
n is an integer from 0 to 10;
   the terminal moiety being attached to the support by at least one linker.

Detection of chemical compounds or molecular complexes in mixtures are typically carried out in the biotechnical field and a skilled addressee would be aware of the wide range of methodologies that are typically utilised in such detection techniques. A vast majority of these techniques utilised binding of the compound or molecular complex of interest to the binding material and it is these techniques that are referred to here. The contacting step may take a number of forms but it typically either involves adding the sample to the binding material or dipping the binding material into the sample in some way.

Once the material has bound in the contacting step, a number of possible steps may be carried out depending on the ultimate detection process to be used. The binding material used in the contacting step may be of any suitable type such as where the binding material forms part of an array. Alternatively, the binding material may be part of a multi-well analytical apparatus. Such array or chip analysis techniques are, broadly speaking, well known and understood in the art. Suitable supports in such applications include glass slides, silicon chips, micro wells, PVDF membranes and magnetic and other micro beads. It is particularly preferred that the support is a glass support.

As would also be clear to a skilled addressee in these applications, the support may have planar architecture such as in a slide or may have alternative architectures. Thus, for example, the support may have engineered micro channels on the surface of the support, or tiny micro-posts may occur on the surface of the support to which the linker is attached. It is preferred, however, that the support is substantially planar.

Once the binding material has been brought into contact with the sample, there are a number of methods well known in the art by which the contacted material can be processed to detect the presence of bound material. In these methods it is preferred that the contacted binding material is washed with a suitable mobile phase in order to remove any unbound material. The mobile phase to be used in the washing will depend on the material bound but is typically chosen so as only to remove unbound material and not to affect the binding interaction. A typical mobile phase is water or an isotonic saline solution.

Following the contacting of the binding material and the washing step (if applicable), there are a number of techniques that may be used to detect the presence of the bound material. The process then preferably involves treating the contacted binding material to determine the presence of the compound or molecular complex. It is preferred, however, that the treating involves treatment of the contacted binding material with a tagged compound which selectively binds to the compound or molecular complex already bound to the binding material. The identity of the tagged compound will depend on the compound or molecular complex being screened or detected for, however, in general, a skilled addressee would be aware of the type of tagged compound that should be used with the compound or molecular complex of interest. Thus, it is well known that if the target molecule is a protein or peptide, for example, the tagged compound should preferably be a tagged ligand or receptor for that protein. With a majority of compounds, a skilled addressee is usually aware of a suitable molecule to use in this step.

When methodology like this is utilised it is then preferred that the binding material is again washed to remove any tagged compound that has not bound to the binding material through the compound or molecular complex being detected. This is usually carried out in order to ensure that false “positives” are not recorded due to residual tagged compounds being present.

Once this has been done, it is preferred that the material is tested for the presence of the tagged compound. In general, the presence of the tagged compound is indicative of the presence of the compound or molecular complex being analysed for. There are a number of suitable tags that may be used as are well known in the art, however, it is preferred that the tag is a radioactive tag, a fluorescent tag, or a chemiluminescent tag. The type of tag to be used will generally depend on the molecule being analysed and the ease of access to the tagged molecule.

A skilled addressee would be aware that fluorescent, radioactive and chemiluminescent tags are the most common tags and methods of testing for the presence of these tags are well known in the art. Any of these methods may be used. In some instances with tags of these types, the method provides the ability to give information not only on the qualitative presence of the compound being detected for, but also may provide quantitative data on the amount of compound present.

In yet an even further aspect the present invention provides a method of analysis of a sample containing a plurality of compounds or molecular complexes, the method comprising:
(a) bringing the sample into contact with a binding material, the binding material comprising:
(i) a support;
(ii) at least one terminal moiety; and
(iii) at least one linker of the formula 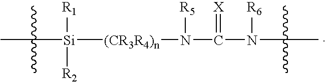
wherein R₁, R₂ may be the same or different and are independently selected from the group consisting of —H, C₁-C₆ alkyl, C₂-C₆ alkenyl, halogen, C₁-C₆ alkoxy, C₂-C₆ alkenoxy and aryloxy, or at least one of R₁ or R₂ when taken together with R₁ or R₂ on an adjacent linker forms a group —O—, wherein said group —O— connects the silicon atom of the linker to the silicon atom of the adjacent linker,
R₃ and R₄ may be the same or different and are independently selected from the group consisting of H, C₁-C₆ alkyl, C₂-C₆ alkenyl and halogen,
R₅ is H or C₁-C₆ alkyl
R₆ is H, C₁-C₆ alkyl, C₂-C₆ alkenyl, aryl, and heteroaryl,
X is O or S,
n is an integer from 0 to 10;
   said terminal moiety being attached to said support by at least one linker;
(b) treating the product of step (a) to separate at least a portion of the components of the sample;
(c) analysing the separated components.

The method of analysis is similar in many respects to the methods of increasing the purity and/or separation of a sample as discussed above and many of the steps will not be discussed again. In general, the first two steps of the process are the same and steps (a) and (b) are therefore preferably conducted in the same way.

The main difference is that rather than collecting eluted fractions (as is the case with increasing the purity), it is preferred in the method of analysis of the sample, that the eluant is analysed immediately to provide an analysis of the components of the sample. This can occur in a number of ways, however, it is typical that it occurs via directing the eluted material from the end of the column to a detection device. The detection device may take any of a number of forms well known in the art, however, it is typically a spectrophotometric device or mass spectrometric device. These devices are well known in the art and it can provide both qualitative and quantitative insight into the components of the sample.

Alternatively, the eluted fractions may be combined to provide pure samples of compounds or molecular complexes of interest which can then be analysed in order to determine the make up of the sample. The analytical techniques used can be any of those well known in the art and may include infrared, mass spectrometry, or NMR, by way of example.

The present invention will now be explained by the following examples.

### EXAMPLES

### Example 1

### Synthesis of Phosphatidylcholine Derivative

### N-Benzyloxycarbonyl-12-aminododecanoic acid (2)

N-(Benzyloxycarbonyloxy)-succinimide (3.24 g, 13 mmol) was added to a solution of 12-aminododecanoic acid (1) (2.15 g, 10 mmol) and triethylamine (1.21 g, 12 mmol) in 100 ml 60% MeOH/H₂O and the mixture stirred at room temperature for 8 hrs under N₂. The solution was then kept at 0° C. for 12 hrs, the resulting white precipitate was filtered and washed with ice-cold 60% MeOH/H₂O). The white residue was vacuum dried over P₂O₅ and further tested with ninhydrin reagent which confirmed the absence of a free amino group. The final product was obtained in 93% yield. m.p. 84-85° C.; HRMS calc'd for (C₂₀H₃₁NO₄Na)⁺:m/z=372.2151, found 372.2136. FTIR (Nujol) 3346, 2922, 2853, 1692, 1684, 1529, 1472, 1274, 1237, 944, 732, 696 cm⁻¹, ¹H NMR (CDCl₃) δ ppm, 1.31 (s br, 14H, —CH₂—(CH₂)₇—CH₂—), 1.49 (qt, 2H, —CH₂—CH₂—COOH), 1.65 (qt, 2H, —NH—CH₂—CH₂—), 2.37 (t, 2H, —CH₂—COOH), 3.19 (q, 2H, —NH—CH₂—CH₂—), 4.81 (s br, 1H, —NH—), 5.12 (s, 2H, —CH₂—C₆H₅), 7.37 (s, 5H, —C₆H₅). ¹³C NMR δ (ppm), 178.5, 156.5, 136.8, 128.5, 128.1, 66.6, 45.7, 41.1, 34.1, 33.5, 29.9, 29.4, 29.2, 29.0, 26.7, 24.7, 8.4.

### N-Benzyloxycarbonyl-12-aminododecanoic acid anhydride (3)

DCC (0.91 g, 4.4 mmol) dissolved in 10 ml dry CCl₄ was added to a stirred solution of (2) (2.80 g, 8 mmol) in 100 ml dry CCl₄ and 50 ml ethanol-free CHCl₃. The solution was stirred at room temperature for 6 hrs under N₂. After the reaction was complete as tested by TLC, the white precipitate was filtered and washed with 4×25 ml CCl₄. The white residues were then added to 200 ml ethanol-free CHCl₃ and kept on ice for 10 min. The CHCl₃ solution was filtered and the white precipitate washed with 3×25 ml ice-cold ethanol-free CHCl₃. The CHCl₃ filtrate was combined and the CHCl₃ solvent was evaporated under low pressure. The white residue was further vacuum dried over P₂O₅ for 24 hrs with 85% yield. m.p. 72-73° C.; HRMS calc'd for (C₄₀H₆₀N₂O₇Na)⁺:m/z=703.4301, found 703,4289. FT-IR (Nujol) 3348, 2922, 2853, 1809, 1742, 1686, 1529, 1472, 1267, 1246, 1228, 1073, 734, 696 cm⁻¹.

### 1,2-Di-O-(N-benzyloxycarbonyl-12-aminododecanoyl)-sn-glycerol-3-O-phosphoryl-choline (4)

sn-Glycero-3-phosphorylcholine (GPC) as its cadmium chloride (CdCl₂) complex (0.44 g, 1 mmol) was dried by repeated azeotropic removal of water with benzene under vacuum. The anhydrous GPC-CdCl₂ complex was washed suspended in 1-ml freshly distilled ethanol-free CHCl₃. Freshly prepared (3) (2.04 g, 3 mmol) and DMAP (0.24 g, 2 mmol) were added to the constantly stirred GPC-CdCl₂ suspension and the flask was flushed with N₂ and then sealed. The suspension was stirred at room temperature in the dark and the reaction was monitored by TLC (eluent CHCl₃:CH₃OH:H₂O=65:25:4 visualisation with Phospray or iodine vapour). After 3 days, the solvent was evaporated under vacuum and the white residue was dissolved in CHCl₃:CH₃OH:H₂O=65:25:2. The solution mixture was then passed through AG 501-X8 resin (BioRad) to remove the CdCl₂ and the resin was further with 3 bed volumes of the same solvent. The eluate was removed under vacuum followed by freeze-drying. The dried product was dissolved in CH₃OH and purified by preparative RP-HPLC (C18) using isocratic elution with solvent CH₃CN:CH₃OH:H₂O=6:3:1 to give the final product in 85% yield. m.p.: 62-63° C.; TLC: R_{f}=0.61 (CHCl₃:CH₃OH:H₂O=65:25:4); HRMS calc'd for (C₄₈H₇₈N₃O₁₂PNa)+: m/z=942.5224, found 942.5221. FT-IR (Nujol): 3341, 2922, 2852, 1723, 1684, 1531, 1472, 1268, 1231, 1090, 1061, 970 cm⁻¹. ¹H NMR (CDCl₃) δ ppm, 1.25 (s br, 28H, —CH₂—(CH₂)₇—CH₂—), 1.48 (qt, 4H, —CH₂—CH₂—COOH), 1.57 (qt, 4H, —NH—CH₂—CH₂—), 2.27 (t, 4H, —CH₂—COOH), 3.15 (q, 4H, —NH—CH₂—CH₂—), 3.34 (s br, 9H, —⁺N(CH₃), 3.78 (s br, 2H, —CH₂—⁺N(CH₃)₃), 3.95 (m, 2H, —CH—CH₂—O—P—), 4.13-4.40 (m, 2H, —CH₂—CH—), 4.31 (s br, 2H, —P—O—CH₂), 4.88 (s br, 2H, —NH—), 5.08 (s, 4H, —CH₂—C₆H₅), 5.21 (qt, 1H, —CH₂—CH—CH₂—) 7.34 (s, 10H, —C₆H₅). ¹³C-NMR δ (ppm), 173.58, 173.22, 156.46, 136.81, 128.55-128.07, 70.66, 70.59, 66.55, 63.43, 63.02, 59.30, 54.58, 41.17, 34.35, 34.16, 30.98-26.77, 24.96. ³¹P-NMR δ (ppm) 0.41.

### 1,2-Di-O-(12-aminododecanoyl)-sn-glycerol-3-O-phosphorylcholine (5)

Freshly prepared (4) (300 mg, 0.325 mmol) was dissolved in 100 ml CH₃OH and to this was added 30 mg of 10% Pd/C. The solution was stirred at room temperature under an atmosphere of H₂. The reaction was monitored by TLC (eluent CHCl₃:CH₃OH:H₂O=65:25:4). The reaction was stopped once all the starting material had been consumed. The mixture was filtered through celite and washed with 3×50 ml CH₃OH. The combined CH₃OH washings were evaporated at 35° C. under vacuum and the final product was vacuum dried over P₂O₅ for 12 hrs. HRMS calc'd for (C₃₂H₆₇O₈N₃P)⁺: m/z=652.4669, found 652.4648. FT-IR (Nujol): 3365, 2918, 2854, 1745, 1652, 1575, 1244, 1174, 1689, 970, 927, 875, 823, 722, 666 cm⁻¹. ¹H NMR (CD₃OD) δ ppm, 1.30 (s br, 28H, —CH₂—(CH₂)₇—CH₂—), 1.59 (qt, 4H, —CH₂—CH₂—COOH), 1.54 (qt, 4H, —NH—CH₂—CH₂—), 2.30 (t, 4H, —CH₂—COOH), 2.74 (m, 4H, —NH—CH₂—CH₂—), 3.22 (s br, 9H, —⁺N(CH₃)₃), 3.89 (s br, 2H, —CH₂ —⁺N(CH₃)₃), 3.99 (m, 2H, —CH—CH₂—O—P—), 4.14-4.41 (m, 2H, —CH₂—CH—), 4.28 (s br, 2H, —P—O—CH₂—), 5.21 (qt, 1H, —CH₂—CH—CH₂—). ¹³C-NMR δ (ppm), 174.95, 174.62, 72.57, 67.95, 64.95, 60.39, 54.73, 41.47, 34.92, 34.82, 31.44-27.46, 25.99. ³¹P-NMR δ (ppm) 0.97.

### Example 2

### Synthesis of Phosphatidic Acid Derivative

### 1,2-di-O-(N-benzyloxycarbonyl-12-aminododecanoyl)-sn-glycero-3-O-phosphate (8)

Dicyclohexylammonium sn-glycero-3-phosphate (370 mg, 1 mmol) was dissolved in 10 ml Milli-Q. The dicyclohexylammonium was converted to the pyridinium salt by passing the aqueous solution of sn-glycero-3-phosphate through pyridinium Dowex-50 ion exchange resin. The resulting residue of the pyridinium salt was rendered anhydrous by repeated evaporation of added anhydrous pyridine (3×20 ml). The dry pyridinium salt of sn-glycero-3-phosphate was re-suspended in 100 ml anhydrous ethanol-free CHCl₃ and sonicated for 10 min. 3 molar equivalents of DMAP (366 mg, 3 mmol) and the freshly prepared N-benzyloxycarbonyl-12-aminododecanoic acid anhydride [3] (2.04 g, 4 mmol) were added to the suspension. The mixture was stirred at room temperature and kept under dark for 72 hours. After the reaction was complete, the solvent was evaporated at 35° C. under reduced pressure and the dried residue was then dissolved in 50 ml CHCl₃ and purified by flash column chromatography with CHCl₃ to remove the fatty acid derivatives, then followed by CHCl₃/CH₃OH=9:1 and CHCl₃/CH₃OH=1:1. The residue was further lyophilized to give the final product with 74% yield. m.p.: 70-71° C.; TLC: Rf=0.83 (CHCl₃:CH₃OH:H₂O=65:25:4), 0.54 (CHCl₃:CH₃OH=1:1); HRMS calculated for (C₄₃H₆₅N₂O₁₂P)⁺: m/z=832.4278, found 832.4263; FT-IR (Nujol): 3341, 2922, 2851, 1726, 1684, 1650, 1558, 1531, 1402, 1321, 1268, 1249, 1231, 1175, 1110, 1051, 943 cm⁻¹. ¹H-NMR (CDCl₃) δ (ppm): 1.24 [—(CH₂)₇—; 28H], 1.56 [—CH₂—CH₂—COO—; 4H], 1.48 [—NH—CH₂—CH₂; 4H], 2.26 [—CH₂—COO—; 4H], 3.17 [—NH—CH₂; 4H], 4.03 [—CH—CH₂—O—P—, 2H], 4.16-4.36 [CH₂—CH—, 2H], 4.89 [—OCO—NH—, 2H], 5.08 [C₆H₅—CH₂—O—, 4H], 5.21 [CH₂—CH—CH₂—, 1H], 7.34 [C₆H₅—CH₂—, 10H]. ¹³C-NMR (CDCl₃) δ (ppm), 173.46, 173.13, 156.76, 136.79, 128.51-128.04, 70.27, 66.52, 63.49, 62.69, 34.24, 34.11, 29.47-26.77, 24.87. ³¹P-NMR (CDCl₃) δ (ppm) 2.48.

### 1,2-di-O-(12-aminododecanoyl)-sn-glycero-3-O-phosphate (9)

The freshly prepared compound (8) (300 mg, 0.360 mmol) was dissolved in a mixture of 20 ml CHCl₃ and 80 ml CH₃OH and 30 mg of 10% Pd/C was added to the stirred solution. The solution was stirred at room temperature under H₂ atmosphere. The reaction was monitored by TLC using solvent CHCl₃:CH₃OH:H₂O=65:25:4. The reaction was stopped after the disappearance of compound (1) on the TLC plate. The mixture was filtered through a fine sintered glass funnel filled with a thick layer of celite and washed with 3×50 ml CH₃OH. The combined CH₃OH washings were evaporated at 35° C. under vacuum and the final product was vacuum dried over P₂O₅ for 12 hr. m.p. 55-56° C.; HRMS calculated for (C₂₇H₅₈N₂O₈P)⁺: m/z=569.3933, found 569.3917. FT-IR (Nujol): 3348, 2922, 2852, 1738, 1270, 1250, 1234, 1179, 1122, 1070, 946 cm⁻¹. ¹H-NMR (CD₃OD) δ (ppm): 1.34 [—(CH₂)₇—; 28H], 1.63 [—CH₂—CH₂—COO—; 4H], 1.68 [—NH—CH₂—CH₂; 4H], 2.35 [—CH₂—COO—; 4H], 2.94 [H₂N—CH₂; 4H], 4.02 [—CH—CH₂—O—P—, 2H], 4.21-4.43 [CH₂—CH—, 2H], 5.23 [CH₂—H—CH₂—, 1H]. ¹³C-NMR (CD₃OD) δ (ppm), 175.00, 174.67, 72.05, 67.31, 64.61, 63.77, 40.83, 35.09, 34.91, 30.53-28.60, 27.47, 25.96. ³¹P-NMR (CD₃OD) δ (ppm) 0.09.

### Example 3

### Synthesis of Phosphatidyl Glycerol

### 1,2-di-O-(N-benzyloxycarbonyl-12-aminododecanoyl)-sn-glycero-3-O-phosphoryl-(−)-2,3-isopropylidene-sn-glycerol (10)

The synthesis of the immobilisable phosphatidylglycerol derivative from the phosphatidylcholine derivative [4] using a trans-phosphatidylation reaction in a biphasic reaction mixture. L-2,3-O-isopropylidene-sn-glycerol (331.0 mg, 2.5 mmol) was dissolved in 10 ml of 100 mM NaOAc and 50 mM CaCl₂ buffer with acetic acid used to adjust the pH to 5.6. The phosphatidylcholine derivative (4) (460.0 mg, 0.5 mmol) dissolved in 10 ml of dichloromethane was added to the buffer mixture. 100 μl of phospholipase D from Streptomyces species (Sigma, P-4912) in which the activity was adjusted to 1 unit/μl with 100 mM NaOAc buffer pH 5.6 was added to the biphasic mixture. The reaction was carried out at 35° C. with high speed stirring. The progress of the transphosphatidylation reaction was monitored by withdrawing a small portion of the mixture every 30 min which was analysed by TLC using the solvent CHCl₃:CH₃OH:H₂O=65:25:2. The reaction was completed after 5 hours. The reaction mixture was separated into each phase by centrifugation at 3000 rpm at 5° C. for 15 min. The upper aqueous phase was withdrawn and the organic phase was further washed with 2×10 ml Milli-Q. The organic solvent was evaporated under vacuum at 35° C. The resulting white solid was then purified by silica gel chromatography. After washing the column with CHCl₃, the final product (3) was eluted using CHCl₃:CH₃OH=2:1 to gave 93% yield as a white solid. m.p.: 62-63° C.; TLC: Rf=0.605 (CHCl₃:CH₃OH:H₂O=65:25:4); HRMS calculated for (C₄₉H₇₆N₂O₁₄P)⁺: m/z=947.5037, found 947.5021. FT-IR (Nujol): 3337, 2922, 2862, 1732, 1685, 1532, 1268, 1248, 1232, 1172, 1109, 1049, 970 cm⁻¹. ¹H-NMR (CDCl₃) δ (ppm): 1.25 [—(CH₂)₇—; 28H], 1.31 and 1.37 [CH₃—C—CH₃; 6H], 1.47 [—CH₂—CH₂—COO—; 4H], 1.57 [—NH—CH₂—CH₂—; 4H], 2.27 [—CH₂—COO—; 4H], 3.15 [—NH—CH₂—; 4H], 3.87 [polar head —P—O—CH₂—CH—, 2H], 3.78-4.00 [polar head —CH₂—CH₂—; 2H], 3.99 [glycerol —CH—CH₂—O—P—, 2H], 4.16-4.41 [glycerol —CH₂—CH—, 2H], 4.27 [polar head —CH₂—CH—CH₂—, 1H], 4.86 [—OCO—NH—, 2H], 5.08 [C₆H₅—CH₂—O—, 4H], 5.28 [glycerol —CH₂—CH—CH₂—, 1H], 7.32 [C₆H₅—CH₂—, 10H]. ¹³C-NMR (CDCl₃) δ (ppm), 173.68 (—COO—), 173.46 (—COO—), 156.45 (—OC—NH—), 136.72 (C₆H₆—CH₂—), 128.51-128.07 (C₆H₅—), 109.40 (solketal quaternary C), 74.74 (polar head —CH—), 70.66 (glycerol —CH—), 66.58 and 66.45 (polar head —CH₂—), 63.95 (glycerol C3), 62.87 (glycerol C1), 41.16, 34.26, 34.08, 29.99-29.12, 26.81 (—C^{β}H₂—), 26.78 (solketal —CH₃), 25.33 (solketal —CH3), 24.84 (ω1 —CH₂—). ³¹P-NMR (CDCl₃) δ (ppm) −2.51.

### 1,2-di-O-(12-aminododecanoyl)-sn-glycero-3-O-phosphoyl-(−)2,3-isopropylidene-sn-glycerol (11)

The freshly prepared compound (10) (300 mg, 0.360 mmol) was dissolved in 50 ml CH₃OH and 30 mg of 10% Pd/C was added to the stirred solution which was stirred at room temperature under H₂ atmosphere. The reaction was monitored by TLC (CHCl₃:CH₃OH:H₂O=65:25:4) and the reaction was stopped after the disappearance of compound (3). The mixture was filtered through a fine scintered glass funnel and washed with 3×50 ml CH₃OH. The combined CH₃OH washings were evaporated at 35° C. under vacuum and the final product was vacuum dried over P₂O₅ for 12 hrs. m.p.: 47-48° C.; HRMS calculated for (C₃₃H₆₄N₂O₁₀P)+: m/z=679.4301, found 679.4293. FT-IR (Nujol): 3343, 2922, 2852, 1740, 1269, 1250, 1245, 1175, 1112, 1060, 945 cm⁻¹. ¹H-NMR (CD₃OD) δ (ppm): 1.31 [—(CH₂)₇—; 28H], 1.29 and 1.38 [CH₃—C—CH₃; 6H], 1.60 [—CH₂—CH₂—COO—; 4H], 1.65 [—NH—CH₂—CH—; 4H], 2.31 [—CH₂—COO—; 4H], 2.91 [H₂N—CH₂—; 4H], 3.90 [polar head —P—O—CH₂—CH—, 2H], 3.79-4.08 [polar head —CH—CH₂—; 2H], 4.02 [glycerol —CH—CH₂—O—P—, 2H], 4.18-4.39 [glycerol —CH₂—CH—, 2H], 4.28 [polar head —CH₂—CH—CH₂—, 1H], 5.21 [glycerol —CH₂—CH—CH₂—, 1H]. ¹³C-NMR (CD₃OD) δ (ppm), 177.92 (—COO—), 177.58 (—COO—), 110.66 (solketal quaternaryC), 76.10 (polar head —CH—), 72.32 (glycerol —CH—), 67.93 and 67.27 (polar head —CH₂—), 65.09 (glycerol C3), 63.52 (glycerol C1), 40.89, 34.95, 30.92-30.06, 28.57 (—C^{β}H₂—), 27.17 (solketal —CH₃), 25.62 (solketal —CH3), 25.92 (ω1 —CH₂—). ³¹P-NMR (CDCl₃) δ (ppm) 0.69.

### Example 4

### Activation of Silica Particles

The ZORBAX 300 RX-SIL 5 μm silica particles (20 g) were suspended in 100 ml isopropanol with the addition of a few drops of HCl to neutralise the NH₃ and sonicated for 10 minutes. The silica suspension was then rotary mixed at medium speed for 24 hrs. The resulting porous silica suspension was then centrifuged at 2000 r.p.m. for 10-15 min. After decanting the clear supernatant, the particles were further washed with 2×100 ml isopropanol and 2×100 ml Milli-Q water each. The porous silica particles were then placed in a round-bottom flask and dried at 120° C. for 24 hrs. Any remaining water was completely removed from the surface of the silica particles by heating at 453K (180° C.) under high vacuum.

### Example 5

### Modification of Silica Particles with 3-isothiocyanatopropyltriethoxysilane (ITCPS)

The dehydrated and activated silica particles (10.0 g) were suspended in freshly distilled anhydrous toluene (150 ml) and ITCPS (1.87 g, 8 mmol) was added to the suspension under vacuum in order to allow the solvent and ligands to penetrate into the surface structure of the porous silica particles. The amount of ITCPS silane used per gram of silica particles was calculated on the basis to achieve a ligand density of 8 μmol/m² support surface which is twice the amount of silane than can theoretically be immobilised based on steric considerations. A small amount of imidazole (about 1%, w/w) was added as catalyst and the mixture was then sonicated for 10 minutes. The reaction mixture was then heated and refluxed for 24 hrs under anhydrous conditions.

The silica suspension was filtered through a 0.22 μm nylon membrane and washed with 3×100 ml toluene followed by 2×100 ml isopropanol and 2×100 ml Milli-Q water. The ITCPS modified porous silica particles were dried at 45° C. under high vacuum for 24 hrs and stored over anhydrous silica gel until usage. The ligand density of the modified porous silica particles was calculated from elemental analysis of the carbon, nitrogen and hydrogen content which is listed in Table 1.

TABLE 1

| | Ligand Density^{a} | | | | | | |
|---|---|---|---|---|---|---|---|
| Coupling | Carbon | | Nitrogen | | Hydrogen | | Average |
| Stage | (μmol/m²) | Mass % | (μmol/m²) | Mass % | (μmol/m²) | Mass % | (μmol/m²) |
| NCS-Si | 1.98 | 0.75 | 1.82 | 0.07 | 1.88 | 0.12 | 1.89 |
| PC-NCS-Si | 0.87 | 1.50 | 0.79 | 0.15 | 0.84 | 0.25 | 0.83 |
| 1st coupling | | | | | | | |
| PC-NCS-Si | 1.02 | 1.76 | 1.27 | 0.24 | 0.94 | 0.28 | 1.08 |
| 2nd coupling | | | | | | | |
| C8 | 3.29 | 1.78 | N.D. | — | 4.41 | 0.46 | 3.85 |

^{a}Average of duplicate analyses.
N.D.: Not detected.

### Example 6

### Immobilisation of ω-Amino Glycerophospholipids onto a Silica Support

The immobilisable PA, PG and PC derivatives were covalently bound to the modified particles via the —NCS groups with the free amino groups accessible at the ω-end of both acyl chains. Each of the phospholipid derivatives (2.4 mmol) was added to the ITCPS-modified silica (3.0 g) in 30 ml methanol and sonicated for 5 minutes. The phospholipid-silica suspension was gently agitated by inverse shaking at room temperature for 48 hrs. The silica particles were then vacuum-filtered through a 0.22 μm nylon membrane and washed with 3×50 ml CH₃OH. The final lipid immobilised silica particles were then vacuum dried at room temperature. The ligand density of the immobilised glycerophospholipids on the porous silica support was determined from elemental analysis of the carbon, hydrogen and nitrogen content of the lipid matrix and listed in Table 1. In order to block the unreacted isothiocyanate groups remaining on the silica surface, the phospholipid immobilised porous silica was further reacted with octylamine, under the same reaction conditions and rinsing steps described above.

The immobilised column chromatographic supports described above were then used in a number of purifications. The mixtures used and the chromatographic conditions used were as follows.

The mixtures used were:

| | |
|---|---|
| Mixture 1: | Ribonuclease A + Cytochrome c + Thermolysin |
| Mixture 2: | Melittin + 21Q melittin analogue (used in Thesis |
| | for Biophysical studies) |

The chromatographic conditions used were:

| | |
|---|---|
| Buffer A: | 0.1% trifluoroacetic acid (TFA) |
| Buffer B: | 0.1% TFA/60% acetonitrile |
| Gradient: | 0-100% buffer B over 30 mins |
| Flow Rate: | 1 ml/min |
| Detection: | 214 nm |

The columns used were:

| | |
|---|---|
| PC: | immobilised phosphatidylcholine |
| PA: | immobilised phosphatidic acid |
| PG: | immobilised phosphatidylglycerol |

### Example 7

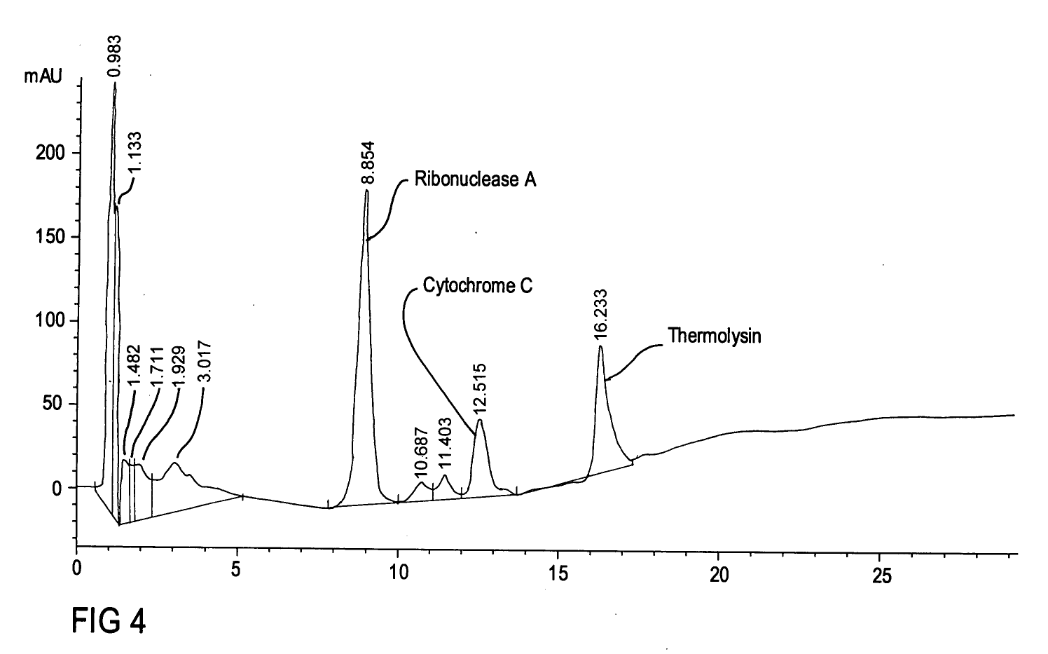

In this example, a column made from the immobilised phosphatidyl choline of example 1 was used to purify a mixture of Ribonuclease A, Cytochrome C and Thermolysin. The chromatographic conditions used were to load the mixture onto the chromatographic support using a solution of 0.1% trifluoroacetic acid in water and then to elute the column with this mixture such that over 30 minutes the mobile phase was changed from 0.1% trifluoroacetic acid to 40% 0.1% trifluoroacetic acid, 60% acetonitrile. The elution rate was constant. The flow rate of the chromatographic column was 1 ml/minute and the detector used had a wave length of 214 nanometres. The output of the detector is shown in FIG. 4.

### Example 8

In this example, a column made from the immobilised phosphatidyl choline of example 4 was used to purify a mixture of Melittin and 21Q. The structures of these materials are as follows:

| | | Molecular |
|---|---|---|
| Solute | Sequence | Mass. |
| Melittin | GIGAVLKVLTTGLPALISWIKRKRQQ-NH₂ | 2847 |
| 21Q | GIGAVLKVLTTGLPALISWQ-NH₂ | 2150 |

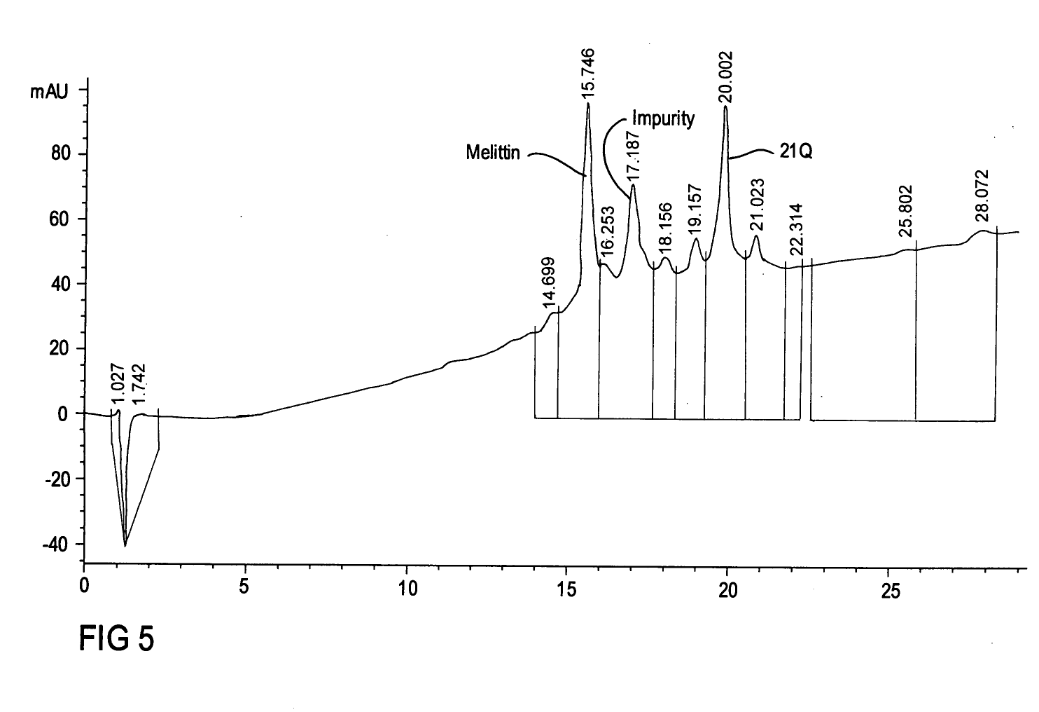

The chromatographic conditions used were to load the mixture onto the chromatographic support using a solution of 0.1% trifluoroacetic acid in water and then to elute the column with this mixture such that over 30 minutes the mobile phase was changed from 0.1% trifluoroacetic acid to 40% 0.1% trifluoroacetic acid, 60% acetonitrile. The elution rate was constant. The flow rate of the chromatographic column was 1 ml/minute and the detector used had a wave length of 214 nanometres. The output of the detector is shown in FIG. 5.

### Example 9

In this example, a column made from the immobilised phosphatidic acid of example 2 was used to purify a mixture of Ribonuclease A, Cytochrome C and Thermolysin.

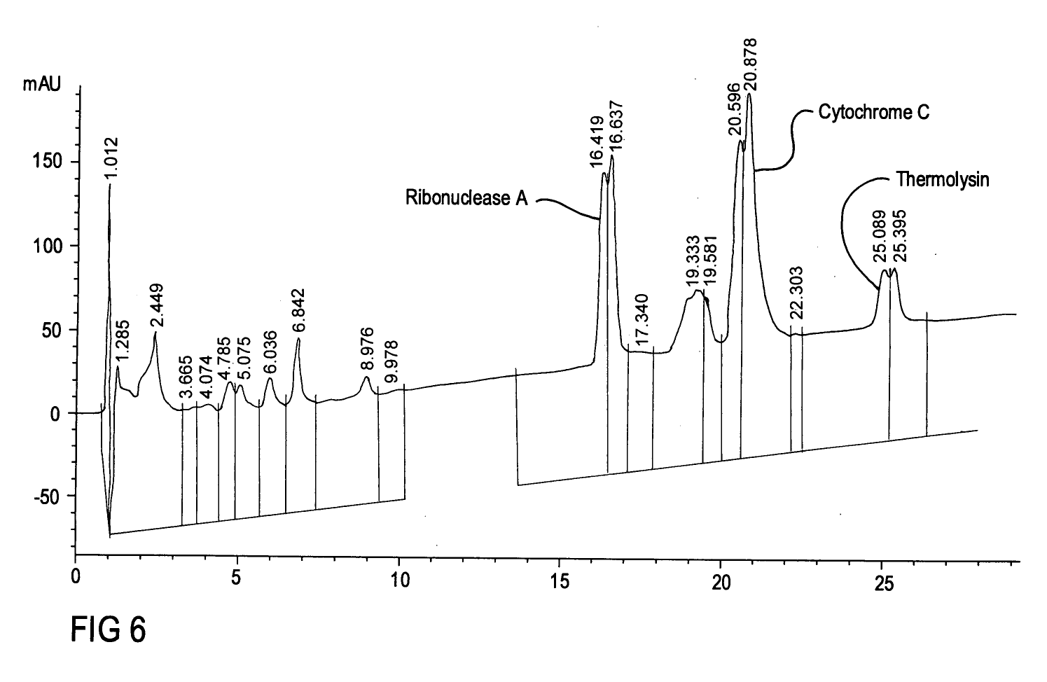

The chromatographic conditions used were to load the mixture onto the chromatographic support using a solution of 0.1% trifluoroacetic acid in water and then to elute the column with this mixture such that over 30 minutes the mobile phase was changed from 0.1% trifluoroacetic acid to 40% 0.1% trifluoroacetic acid, 60% acetonitrile. The elution rate was constant. The flow rate of the chromatographic column was 1 ml/minute and the detector used had a wave length of 214 nanometres. The output of the detector is shown in FIG. 6.

### Example 10

In this example, a column made from the Immobilised phosphatidic acid of example 2 was used to purify a mixture of Melittin and 21Q. The structures are as follows:

| | | Molecular |
|---|---|---|
| Solute | Sequence | Mass. |
| Melittin | GIGAVLKVLTTGLPALISWIKRKRQQ-NH₂ | 2847 |
| 21Q | GIGAVLKVLTTGLPALISWQ-NH₂ | 2150 |

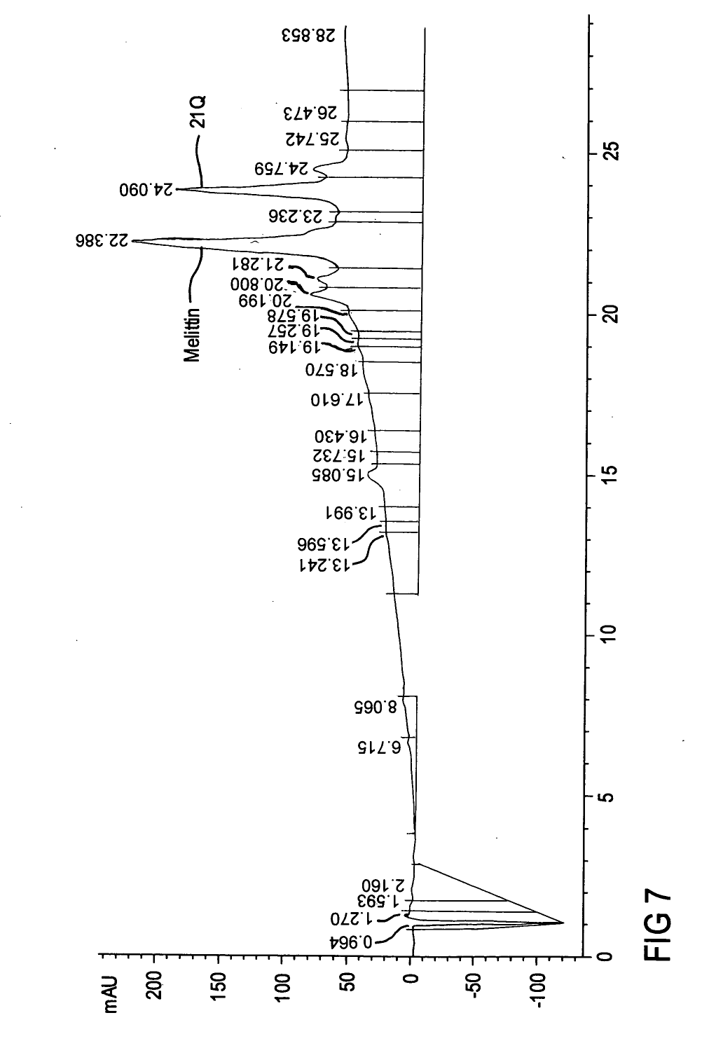

The chromatographic conditions used were to load the mixture onto the chromatographic support using a solution of 0.1% trifluoroacetic acid in water and then to elute the column with this mixture such that over 30 minutes the mobile phase was changed from 0.1% trifluoroacetic acid to 40% 0.1% trifluoroacetic acid, 60% acetonitrile. The elution rate was constant. The flow rate of the chromatographic column was 1 ml/minute and the detector used had a wave length of 214 nanometres. The output of the detector is shown in FIG. 7.

### Example 11

In this example, a column made from the immobilised phosphatidyl glycerol of example 3 was used to purify a mixture of Ribonuclease A, Cytochrome C and Thermolysin.

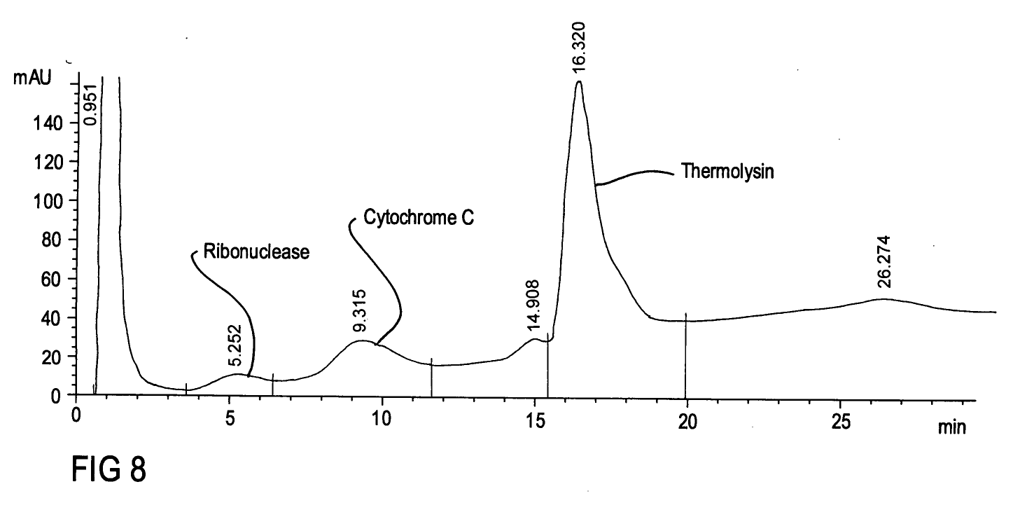

The chromatographic conditions used were to load the mixture onto the chromatographic support using a solution of 0.1% trifluoroacetic acid in water and then to elute the column with this mixture such that over 30 minutes the mobile phase was changed from 0.1% trifluoroacetic acid to 40% 0.1% trifluoroacetic acid, 60% acetonitrile. The elution rate was constant. The flow rate of the chromatographic column was 1 ml/minute and the detector used had a wave length of 214 nanometres. The output of the detector is shown in FIG. 8.

### Example 12

In this example, a column made from the immobilised phosphatidic acid of example 3 was used to purify a mixture of Melittin and 21Q. The structures are as follows:

| | | Molecular |
|---|---|---|
| Solute | Sequence | Mass. |
| Melittin | GIGAVLKVLTTGLPALISWIKRKRQQ-NH₂ | 2847 |
| 21Q | GIGAVLKVLTTGLPALISWQ-NH₂ | 2150 |

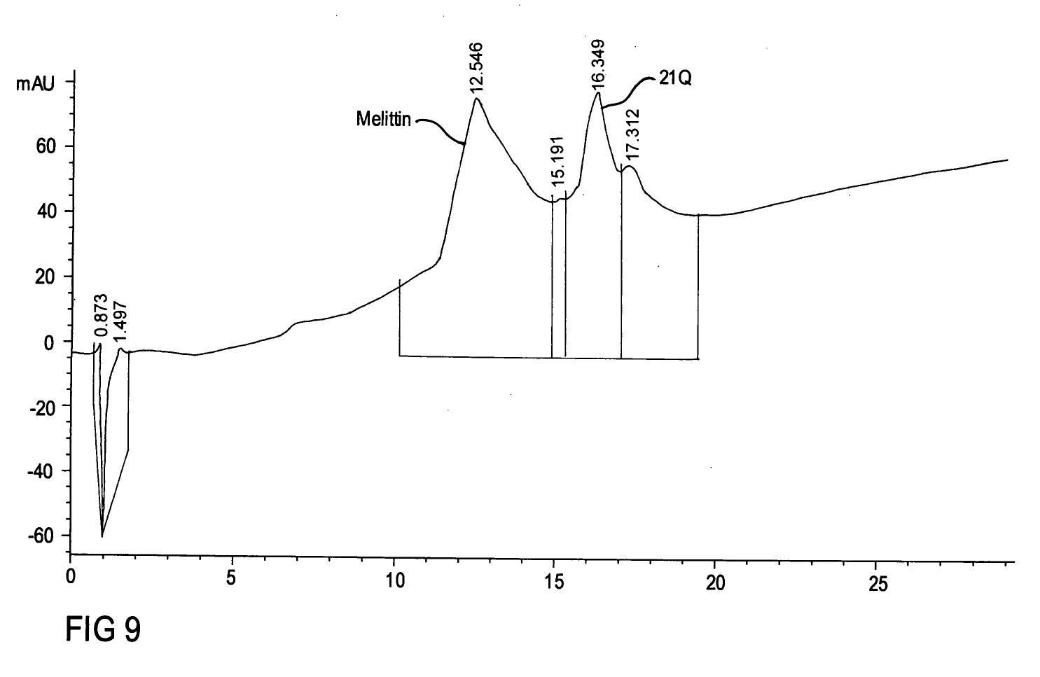

The chromatographic conditions used were to load the mixture onto the chromatographic support using a solution of 0.1% trifluoroacetic acid in water and then to elute the column with this mixture such that over 30 minutes the mobile phase was changed from 0.1% trifluoroacetic acid to 40% 0.1% trifluoroacetic acid, 60% acetonitrile. The elution rate was constant. The flow rate of the chromatographic column was 1 ml/minute and the detector used had a wave length of 214 nanometres. The output of the detector is shown in FIG. 9.

Finally, it is to be understood that various other modifications and/or alterations may be made without departing from the spirit of the present invention as outlined herein.

1. A method of separating a compound or molecular complex on the basis of the ability of the compound or molecular complex to associate with a binding material, from compounds or molecular complexes having different association characteristics, said method comprising:
(a) bringing a sample containing said compound or molecular complex into contact with a binding material, the binding material comprising:
(i) a support,
(ii) at least one terminal moiety selected from the group consisting of lecithins, lysolecithins, cephalins, sphingomyelin, cardiolipin, glycolipids, gangliosides, cerebrosides and phospholipids, and
(iii) at least one linker of the formula 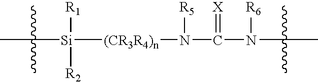
wherein R₁ and R₂ may be the same or different and are independently selected from the group consisting of: H, OH, C₁-C₆ alkyl, C₂-C₆ alkenyl, halogen, C₁-C₆ alkoxy, C₂-C₆ alkenyloxy and aryloxy, or R₁ or R₂ when taken together with an R₁ or R₂ on an adjacent linker forms a group of formula —O—, wherein said group —O— connects the silicon atom of the linker to the silicon atom of the adjacent linker,
R₃ and R₄ may be the same or different and are independently selected from the group consisting of H, C₁-C₆ alkyl, C₂-C₆ alkenyl and halogen,
R₅ is H or C₁-C₆ alkyl,
R₆ is H, C₁-C₆ alkyl, C₂-C₆ alkenyl, aryl and heteroaryl,
X is O or S,
n is an integer from 0 to 10,
the terminal moiety being bound to the support via at least one linker,
(b) treating the product of step (a) to separate the components of the sample on the basis of their ability to associate with the binding material.

2. A method according to claim 1, wherein the linker is of the formula: 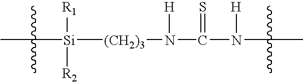
wherein R₁ and R₂ are as defined in claim 1.

3. A method according to claim 1, wherein each of said terminal moieties is independently selected from the group consisting of phosphatidylcholine, phosphatidylethanolamine, N-methyl phosphatidylethanolamine, N,N-dimethylphosphatidylethanolamine, phosphatidic acid, phosphatidylserine, phosphatidylinositol and phosphatidylglycerol or derivatives thereof.

4. A method according to claim 1, wherein said binding material is a chromatographic material loaded within a chromatographic column and said contacting involving loading said sample containing the compound or molecular complex onto said column.

5. A method according to claim 1, wherein said sample contains proteins, peptides, or complexes thereof.

6. A method according to claim 5, wherein the proteins are membrane proteins.

7. A method of detection of a compound or molecular complex in a sample containing said compound or molecular complex, said method comprising:
(a) bringing a sample containing said compound or molecular complex into contact with a binding material, the binding material comprising:
(i) a support;
(ii) at least one terminal moiety consisting of lecithins, lysolecithins, cephalins, sphingomyelin, cardiolipin, glycolipids, gangliosides, cerebrosides and phospholipids; and
(iii) at least one linker of the formula 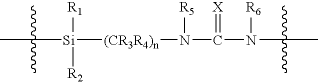
wherein R₁, R₂ may be the same or different and are independently selected from the group consisting of: H, OH, C₁-C₆ alkyl, C₂-C₆ alkenyl, halogen, C₁-C₆ alkoxy, C₂-C₆ alkenyloxy and aryloxy, or R₁ or R₂ when taken together with an R₁ or R₂ on an adjacent linker forms a group of formula —O—, wherein said group —O— connects the silicon atom of the linker to the silicon atom of the adjacent linker,
R₃ and R₄ may be the same or different and are independently selected from the group consisting of H, C₁-C₆ alkyl, C₂-C₆ alkenyl and halogen,
R₅ is H or C₁-C₆ alkyl
R₆ is H, C₁-C₆ alkyl, C₂-C₆ alkenyl, aryl, and heteroaryl,
X is O or S,
n is an integer from 0 to 10;
the terminal moiety being attached to the support by at least one linker.

8. A method according to claim 7 where the linker is of the formula: 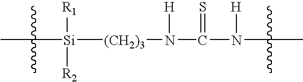
wherein R₁ and R₂ are as defined in claim 1.

9. A method according to claim 7, wherein each of said terminal moieties is independently selected from the group consisting of phosphatidylcholine, phosphatidylethanolamine, N-methyl phosphatidylethanolamine, N,N-dimethylphosphatidylethanolamine, phosphatidic acid, phosphatidylserine, phosphatidylinositol and phosphatidylglycerol or derivatives thereof.

10. A method according to claim 7, wherein each of the terminal moieties is independently selected from the group consisting of phosphatidic acid, phosphatidylglycerol and phosphatidylcholine.

11. A method according to claim 7, wherein said support is the solid support of an array.

12. A method according to claim 7, wherein said sample contains proteins, peptides, or complexes thereof.

13. A method according to claim 12, wherein the proteins are membrane proteins.

14. A method of detection according to claim 7, wherein said method further comprises treating the contacted binding material to determine the presence of the compound or molecular complex.

15. The method according to claim 14, wherein said treating comprises treatment with a tagged compound which selectively binds to said compound or molecular complex.

16. A method according to claim 15, wherein said method further comprises testing said binding material for the presence of said tagged compound, the presence of said tagged compound being indicative of the presence of said compound or molecular complex.

17. A method according to claim 16, wherein said tag is a radioactive tag, a fluorescent tag or a chemiluminescent tag.

18. A method of increasing the purity of a compound or molecular complex from an impure sample containing said compound or molecular complex, the method comprising:
(a) bringing said sample into contact with a binding material, the binding material comprising:
(i) a support;
(ii) at least one terminal moiety selected from the group consisting of lecithins, lysolecithins, cephalins, sphingomyelin, cardiolipin, glycolipids, gangliosides, cerebrosides and phospholipids; and
(iii) at least one linker of the formula 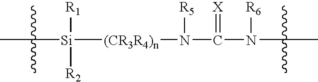
wherein R₁, R₂ may be the same or different and are independently selected from the group consisting of: —H, C₁-C₆ alkyl, C₂-C₆ alkenyl, halogen, C₁-C₆ alkoxy, C₂-C₆ alkenoxy and aryloxy, or at least one of R₁ or R₂ when taken together with R₁ or R₂ on an adjacent linker forms a group —O—, wherein said group —O— connects the silicon atom of the linker to the silicon atom of the adjacent linker,
R₃ and R₄ may be the same or different and are independently selected from the group consisting of H, C₁-C₆ alkyl, C₂-C₆ alkenyl and halogen,
R₅ is H or C₁-C₆ alkyl
R₆ is H, C₁-C₆ alkyl, C₂-C₆ alkenyl, aryl, and heteroaryl,
X is O or S,
n is an integer from 0 to 10;
said terminal moiety being attached to said support by at least one linker;
(b) treating the product of step (a) to separate the compound or molecular complex from at least a portion of the other components in the ample;
(c) recovering at least a portion of the compound or molecular complex.

19. A method according to claim 18 where the linker is of the formula: 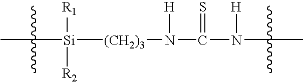
wherein R₁ and R₂ are as defined in claim 1.

20. A method according to claim 18, wherein each of said terminal moieties is independently selected from the group consisting of phosphatidylcholine, phosphatidylethanolamine, N-methyl phosphatidylethanolamine, N,N-dimethylphosphatidylethanolamine, phosphatidic acid, phosphatidylserine, phosphatidylinositol and phosphatidylglycerol or derivatives thereof.

21. A method according to claim 20, wherein each of the terminal moieties is independently selected from the group consisting of phosphatidic acid, phosphatidylglycerol and phosphatidylcholine.

22. A method according to claim 18, wherein said sample contains proteins, peptides, or complexes thereof.

23. A method according to claim 22, wherein the proteins are membrane proteins.

24. A method according to claim 18, wherein said binding material is a chromatographic material loaded within a chromatographic column and said step (b) comprises elution of the column with a mobile phase followed by collection of eluted fractions.

25. A method according to claim 24, wherein said elution involves the use of a solvent gradient.

26. A method according to claim 24, wherein said mobile phase is selected from the group consisting of water, hydrocarbons, esters, alkyl esters, nitrites, alcohols, acids, aqueous solutions of acids and mixtures thereof.

27. A method according to claim 24, wherein said mobile phase is selected from the group consisting of water, ethyl acetate, acetonitrile, ethanol, methanol, and aqueous solutions of trifluoro acetic acid.

28. A method according to claim 24, wherein said recovery comprises combination of eluted fractions containing the same component followed by the removal of the mobile phase to produce the purified compound or molecular complex.

29. A method according to claim 18, wherein said step (b) comprises washing the contacted binding material to remove unbound material.

30. A method according to claim 29, wherein said step (c) comprises treating contacted binding material to remove the bound material.

31. A method of claim 18 wherein, after subjection to the method, the recovered compound or molecular complex has a purity of at least 80%.

32. A method according to claim 31 wherein, after subjection to the method, the recovered compound or molecular complex has a purity of at least 90%.

33. A method according to claim 18, wherein the compound or molecular complex is recovered in substantially pure form.

34. A method of analysis of a sample containing a plurality of compounds or molecular complexes, the method comprising:
(a) bringing the sample into contact with a binding material, the binding material comprising:
(i) a support;
(ii) at least one terminal moiety selected from the group consisting of lecithins, lysolecithins, cephalins, sphingomyelin, cardiolipin, glycolipids, gangliosides, cerebrosides and phospholipids; and
(iii) at least one linker of the formula 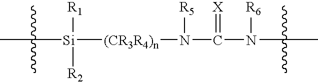
wherein R₁, R₂ may be the same or different and are independently selected from the group consisting of —H, C₁-C₆ alkyl, C₂-C₆ alkenyl, halogen, C₁-C₆ alkoxy, C₂-C₆ alkenoxy and aryloxy, or at least one of R₁ or R₂ when taken together with R₁ or R₂ on an adjacent linker forms a group —O—, wherein said group —O— connects the silicon atom of the linker to the silicon atom of the adjacent linker,
R₃ and R₄ may be the same or different and are independently selected from the group consisting of H, C₁-C₆ alkyl, C₂-C₆ alkenyl and halogen,
R₅ is H or C₁-C₆ alkyl
R₆ is H, C₁-C₆ alkyl, C₂-C₆ alkenyl, aryl, and heteroaryl,
X is O or S,
n is an integer from 0 to 10;
said terminal moiety being attached to said support by at least one linker;
(b) separating the components of the sample;
(c) analysing the separated components.

35. A method according to claim 34, wherein said binding material is a chromatographic support material loaded within a chromatographic column and said step (b) comprises elution of the column with a mobile phase.

36. A method according to claim 35, wherein said elution involves the use of a solvent gradient.

37. A method according to claim 35, wherein said mobile phase is selected from the group consisting of water, hydrocarbons, esters, alkyl esters, nitrites, alcohols, acids, aqueous solutions of acids and mixtures thereof.

38. A method according to claim 35, wherein said mobile phase is selected from the group consisting of water, ethyl acetate, acetonitrile, ethanol, methanol, and aqueous solutions of trifluoro acetic acid.

39. A method according to claim 34, wherein fractions of the eluent are collected and then analysed in step (c).

40. A method according to claim 39, wherein said analysis is conducted using a spectrophotometric technique or by mass spectrometry.

41. A method according to claim 18, wherein said sample contains proteins, peptides, or complexes thereof.

42. A method according to claim 41, wherein the proteins are membrane proteins.

43. A method according to claim 34, wherein said step (b) comprises washing the contacted binding material to remove unbound material.

44. A method according to claim 43, wherein said step (c) comprises treating the contacted binding material to remove the bound material and subjecting the material thus obtained to analysis.

45. A method according to claim 44, wherein said removed bound material is subjected to analysis using a spectrophotometric technique or by mass spectrometry.


SEQUENCE LISTING (2 sequences)

SEQ ID NO: 1 - protein, 26 aa, Homo sapiens
GIGAVLKVLTTGLPALISWIKRKRQQ

SEQ ID NO: 2 - protein, 20 aa, Homo sapiens
GIGAVLKVLTTGLPALISWQ